SEQUENCE LISTING (200 sequences)

SEQ ID NO: 1 - protein, 15 aa, Artificial Sequence
  LMP2a epitope
MGSLEMVPMGAGPPS

SEQ ID NO: 2 - protein, 15 aa, Artificial Sequence
  LMP2a epitope
EMVPMGAGPPSPGGD

SEQ ID NO: 3 - protein, 15 aa, Artificial Sequence
  LMP2a epitope
MGAGPPSPGGDPDGD

SEQ ID NO: 4 - protein, 15 aa, Artificial Sequence
  LMP2a epitope
PPSPGGDPDGDDGGN

SEQ ID NO: 5 - protein, 15 aa, Artificial Sequence
  LMP2a epitope
GGDPDGDDGGNNSQY

SEQ ID NO: 6 - protein, 15 aa, Artificial Sequence
  LMP2a epitope
DGDDGGNNSQYPSAS

SEQ ID NO: 7 - protein, 15 aa, Artificial Sequence
  LMP2a epitope
GGNNSQYPSASGSYG

SEQ ID NO: 8 - protein, 15 aa, Artificial Sequence
  LMP2a epitope
SQYPSASGSYGNTPT

SEQ ID NO: 9 - protein, 15 aa, Artificial Sequence
  LMP2a epitope
SASGSYGNTPTPPND

SEQ ID NO: 10 - protein, 15 aa, Artificial Sequence
  LMP2a epitope
SYGNTPTPPNDEERE

SEQ ID NO: 11 - protein, 15 aa, Artificial Sequence
  LMP2a epitope
TPTPPNDEERESNEE

SEQ ID NO: 12 - protein, 15 aa, Artificial Sequence
  LMP2a epitope
PNDEERESNEEPPPP

SEQ ID NO: 13 - protein, 15 aa, Artificial Sequence
  LMP2a epitope
ERESNEEPPPPYEDL

SEQ ID NO: 14 - protein, 15 aa, Artificial Sequence
  LMP2a epitope
NEEPPPPYEDLDWGN

SEQ ID NO: 15 - protein, 15 aa, Artificial Sequence
  LMP2a epitope
PPPYEDLDWGNGDRH

SEQ ID NO: 16 - protein, 15 aa, Artificial Sequence
  LMP2a epitope
EDLDWGNGDRHSDYQ

SEQ ID NO: 17 - protein, 15 aa, Artificial Sequence
  LMP2a epitope
WGNGDRHSDYQPLGN

SEQ ID NO: 18 - protein, 15 aa, Artificial Sequence
  LMP2a epitope
DRHSDYQPLGNQDPS

SEQ ID NO: 19 - protein, 15 aa, Artificial Sequence
  LMP2a epitope
DYQPLGNQDPSLYLG

SEQ ID NO: 20 - protein, 15 aa, Artificial Sequence
  LMP2a epitope
LGNQDPSLYLGLQHD

SEQ ID NO: 21 - protein, 15 aa, Artificial Sequence
  LMP2a epitope
DPSLYLGLQHDGNDG

SEQ ID NO: 22 - protein, 15 aa, Artificial Sequence
  LMP2a epitope
YLGLQHDGNDGLPPP

SEQ ID NO: 23 - protein, 15 aa, Artificial Sequence
  LMP2a epitope
QHDGNDGLPPPPYSP

SEQ ID NO: 24 - protein, 15 aa, Artificial Sequence
  LMP2a epitope
NDGLPPPPYSPRDDS

SEQ ID NO: 25 - protein, 15 aa, Artificial Sequence
  LMP2a epitope
PPPPYSPRDDSSQHI

SEQ ID NO: 26 - protein, 15 aa, Artificial Sequence
  LMP2a epitope
YSPRDDSSQHIYEEA

SEQ ID NO: 27 - protein, 15 aa, Artificial Sequence
  LMP2a epitope
DDSSQHIYEEAGRGS

SEQ ID NO: 28 - protein, 15 aa, Artificial Sequence
  LMP2a epitope
QHIYEEAGRGSMNPV

SEQ ID NO: 29 - protein, 15 aa, Artificial Sequence
  LMP2a epitope
EEAGRGSMNPVCLPV

SEQ ID NO: 30 - protein, 15 aa, Artificial Sequence
  LMP2a epitope
RGSMNPVCLPVIVAP

SEQ ID NO: 31 - protein, 15 aa, Artificial Sequence
  LMP2a epitope
NPVCLPVIVAPYLFW

SEQ ID NO: 32 - protein, 15 aa, Artificial Sequence
  LMP2a epitope
LPVIVAPYLFWLAAI

SEQ ID NO: 33 - protein, 15 aa, Artificial Sequence
  LMP2a epitope
VAPYLFWLAAIAASC

SEQ ID NO: 34 - protein, 15 aa, Artificial Sequence
  LMP2a epitope
LFWLAAIAASCFTAS

SEQ ID NO: 35 - protein, 15 aa, Artificial Sequence
  LMP2a epitope
AAIAASCFTASVSTV

SEQ ID NO: 36 - protein, 15 aa, Artificial Sequence
  LMP2a epitope
ASCFTASVSTVVTAT

SEQ ID NO: 37 - protein, 15 aa, Artificial Sequence
  LMP2a epitope
TASVSTVVTATGLAL

SEQ ID NO: 38 - protein, 15 aa, Artificial Sequence
  LMP2a epitope
STVVTATGLALSLLL

SEQ ID NO: 39 - protein, 15 aa, Artificial Sequence
  LMP2a epitope
TATGLALSLLLLAAV

SEQ ID NO: 40 - protein, 15 aa, Artificial Sequence
  LMP2a epitope
LALSLLLLAAVASSY

SEQ ID NO: 41 - protein, 15 aa, Artificial Sequence
  LMP2a epitope
LLLLAAVASSYAAAQ

SEQ ID NO: 42 - protein, 15 aa, Artificial Sequence
  LMP2a epitope
AAVASSYAAAQRKLL

SEQ ID NO: 43 - protein, 15 aa, Artificial Sequence
  LMP2a epitope
SSYAAAQRKLLTPVT

SEQ ID NO: 44 - protein, 15 aa, Artificial Sequence
  LMP2a epitope
AAQRKLLTPVTVLTA

SEQ ID NO: 45 - protein, 15 aa, Artificial Sequence
  LMP2a epitope
KLLTPVTVLTAVVTF

SEQ ID NO: 46 - protein, 15 aa, Artificial Sequence
  LMP2a epitope
PVTVLTAVVTFFAIC

SEQ ID NO: 47 - protein, 15 aa, Artificial Sequence
  LMP2a epitope
LTAVVTFFAICLTWR

SEQ ID NO: 48 - protein, 15 aa, Artificial Sequence
  LMP2a epitope
VTFFAICLTWRIEDP

SEQ ID NO: 49 - protein, 15 aa, Artificial Sequence
  LMP2a epitope
AICLTWRIEDPPFNS

SEQ ID NO: 50 - protein, 15 aa, Artificial Sequence
  LMP2a epitope
TWRIEDPPFNSLLFA

SEQ ID NO: 51 - protein, 15 aa, Artificial Sequence
  LMP2a epitope
EDPPFNSLLFALLAA

SEQ ID NO: 52 - protein, 15 aa, Artificial Sequence
  LMP2a epitope
FNSLLFALLAAAGGL

SEQ ID NO: 53 - protein, 15 aa, Artificial Sequence
  LMP2a epitope
LFALLAAAGGLQGIY

SEQ ID NO: 54 - protein, 15 aa, Artificial Sequence
  LMP2a epitope
LAAAGGLQGIYVLVM

SEQ ID NO: 55 - protein, 15 aa, Artificial Sequence
  LMP2a epitope
GGLQGIYVLVMLVLL

SEQ ID NO: 56 - protein, 15 aa, Artificial Sequence
  LMP2a epitope
GIYVLVMLVLLILAY

SEQ ID NO: 57 - protein, 15 aa, Artificial Sequence
  LMP2a epitope
LVMLVLLILAYRRRW

SEQ ID NO: 58 - protein, 15 aa, Artificial Sequence
  LMP2a epitope
VLLILAYRRRWRRLT

SEQ ID NO: 59 - protein, 15 aa, Artificial Sequence
  LMP2a epitope
LAYRRRWRRLTVCGG

SEQ ID NO: 60 - protein, 15 aa, Artificial Sequence
  LMP2a epitope
RRWRRLTVCGGIMFL

SEQ ID NO: 61 - protein, 15 aa, Artificial Sequence
  LMP2a epitope
RLTVCGGIMFLACVL

SEQ ID NO: 62 - protein, 15 aa, Artificial Sequence
  LMP2a epitope
CGGIMFLACVLVLIV

SEQ ID NO: 63 - protein, 15 aa, Artificial Sequence
  LMP2a epitope
MFLACVLVLIVDAVL

SEQ ID NO: 64 - protein, 15 aa, Artificial Sequence
  LMP2a epitope
CVLVLIVDAVLQLSP

SEQ ID NO: 65 - protein, 15 aa, Artificial Sequence
  LMP2a epitope
LIVDAVLQLSPLLGA

SEQ ID NO: 66 - protein, 15 aa, Artificial Sequence
  LMP2a epitope
AVLQLSPLLGAVTVV

SEQ ID NO: 67 - protein, 15 aa, Artificial Sequence
  LMP2a epitope
LSPLLGAVTVVSMTL

SEQ ID NO: 68 - protein, 15 aa, Artificial Sequence
  LMP2a epitope
LGAVTVVSMTLLLLA

SEQ ID NO: 69 - protein, 15 aa, Artificial Sequence
  LMP2a epitope
TVVSMTLLLLAFVLW

SEQ ID NO: 70 - protein, 15 aa, Artificial Sequence
  LMP2a epitope
MTLLLLAFVLWLSSP

SEQ ID NO: 71 - protein, 15 aa, Artificial Sequence
  LMP2a epitope
LLAFVLWLSSPGGLG

SEQ ID NO: 72 - protein, 15 aa, Artificial Sequence
  LMP2a epitope
VLWLSSPGGLGTLGA

SEQ ID NO: 73 - protein, 15 aa, Artificial Sequence
  LMP2a epitope
SSPGGLGTLGAALLT

SEQ ID NO: 74 - protein, 15 aa, Artificial Sequence
  LMP2a epitope
GLGTLGAALLTLAAA

SEQ ID NO: 75 - protein, 15 aa, Artificial Sequence
  LMP2a epitope
LGAALLTLAAALALL

SEQ ID NO: 76 - protein, 15 aa, Artificial Sequence
  LMP2a epitope
LLTLAAALALLASLI

SEQ ID NO: 77 - protein, 15 aa, Artificial Sequence
  LMP2a epitope
AAALALLASLILGTL

SEQ ID NO: 78 - protein, 15 aa, Artificial Sequence
  LMP2a epitope
ALLASLILGTLNLTT

SEQ ID NO: 79 - protein, 15 aa, Artificial Sequence
  LMP2a epitope
SLILGTLNLTTMFLL

SEQ ID NO: 80 - protein, 15 aa, Artificial Sequence
  LMP2a epitope
GTLNLTTMFLLMLLW

SEQ ID NO: 81 - protein, 15 aa, Artificial Sequence
  LMP2a epitope
LTTMFLLMLLWTLVV

SEQ ID NO: 82 - protein, 15 aa, Artificial Sequence
  LMP2a epitope
FLLMLLWTLVVLLIC

SEQ ID NO: 83 - protein, 15 aa, Artificial Sequence
  LMP2a epitope
LLWTLVVLLICSSCS

SEQ ID NO: 84 - protein, 15 aa, Artificial Sequence
  LMP2a epitope
LVVLLICSSCSSCPL

SEQ ID NO: 85 - protein, 15 aa, Artificial Sequence
  LMP2a epitope
LICSSCSSCPLTKIL

SEQ ID NO: 86 - protein, 15 aa, Artificial Sequence
  LMP2a epitope
SCSSCPLTKILLARL

SEQ ID NO: 87 - protein, 15 aa, Artificial Sequence
  LMP2a epitope
CPLTKILLARLFLYA

SEQ ID NO: 88 - protein, 15 aa, Artificial Sequence
  LMP2a epitope
KILLARLFLYALALL

SEQ ID NO: 89 - protein, 15 aa, Artificial Sequence
  LMP2a epitope
ARLFLYALALLLLAS

SEQ ID NO: 90 - protein, 15 aa, Artificial Sequence
  LMP2a epitope
LYALALLLLASALIA

SEQ ID NO: 91 - protein, 15 aa, Artificial Sequence
  LMP2a epitope
ALLLLASALIAGGSI

SEQ ID NO: 92 - protein, 15 aa, Artificial Sequence
  LMP2a epitope
LASALIAGGSILQTN

SEQ ID NO: 93 - protein, 15 aa, Artificial Sequence
  LMP2a epitope
LIAGGSILQTNFKSL

SEQ ID NO: 94 - protein, 15 aa, Artificial Sequence
  LMP2a epitope
GSILQTNFKSLSSTE

SEQ ID NO: 95 - protein, 15 aa, Artificial Sequence
  LMP2a epitope
QTNFKSLSSTEFIPN

SEQ ID NO: 96 - protein, 15 aa, Artificial Sequence
  LMP2a epitope
KSLSSTEFIPNLFCM

SEQ ID NO: 97 - protein, 15 aa, Artificial Sequence
  LMP2a epitope
STEFIPNLFCMLLLI

SEQ ID NO: 98 - protein, 15 aa, Artificial Sequence
  LMP2a epitope
IPNLFCMLLLIVAGI

SEQ ID NO: 99 - protein, 15 aa, Artificial Sequence
  LMP2a epitope
FCMLLLIVAGILFIL

SEQ ID NO: 100 - protein, 15 aa, Artificial Sequence
  LMP2a epitope
LLIVAGILFILAILT

SEQ ID NO: 101 - protein, 15 aa, Artificial Sequence
  LMP2a epitope
AGILFILAILTEWGS

SEQ ID NO: 102 - protein, 15 aa, Artificial Sequence
  LMP2a epitope
FILAILTEWGSGNRT

SEQ ID NO: 103 - protein, 15 aa, Artificial Sequence
  LMP2a epitope
ILTEWGSGNRTYGPV

SEQ ID NO: 104 - protein, 15 aa, Artificial Sequence
  LMP2a epitope
WGSGNRTYGPVFMCL

SEQ ID NO: 105 - protein, 15 aa, Artificial Sequence
  LMP2a epitope
NRTYGPVFMCLGGLL

SEQ ID NO: 106 - protein, 15 aa, Artificial Sequence
  LMP2a epitope
GPVFMCLGGLLTMVA

SEQ ID NO: 107 - protein, 15 aa, Artificial Sequence
  LMP2a epitope
MCLGGLLTMVAGAVW

SEQ ID NO: 108 - protein, 15 aa, Artificial Sequence
  LMP2a epitope
GLLTMVAGAVWLTVM

SEQ ID NO: 109 - protein, 15 aa, Artificial Sequence
  LMP2a epitope
MVAGAVWLTVMTNTL

SEQ ID NO: 110 - protein, 15 aa, Artificial Sequence
  LMP2a epitope
AVWLTVMTNTLLSAW

SEQ ID NO: 111 - protein, 15 aa, Artificial Sequence
  LMP2a epitope
TVMTNTLLSAWILTA

SEQ ID NO: 112 - protein, 15 aa, Artificial Sequence
  LMP2a epitope
NTLLSAWILTAGFLI

SEQ ID NO: 113 - protein, 15 aa, Artificial Sequence
  LMP2a epitope
SAWILTAGFLIFLIG

SEQ ID NO: 114 - protein, 15 aa, Artificial Sequence
  LMP2a epitope
LTAGFLIFLIGFALF

SEQ ID NO: 115 - protein, 15 aa, Artificial Sequence
  LMP2a epitope
FLIFLIGFALFGVIR

SEQ ID NO: 116 - protein, 15 aa, Artificial Sequence
  LMP2a epitope
LIGFALFGVIRCCRY

SEQ ID NO: 117 - protein, 15 aa, Artificial Sequence
  LMP2a epitope
ALFGVIRCCRYCCYY

SEQ ID NO: 118 - protein, 15 aa, Artificial Sequence
  LMP2a epitope
VIRCCRYCCYYCLTL

SEQ ID NO: 119 - protein, 15 aa, Artificial Sequence
  LMP2a epitope
CRYCCYYCLTLESEE

SEQ ID NO: 120 - protein, 15 aa, Artificial Sequence
  LMP2a epitope
CYYCLTLESEERPPT

SEQ ID NO: 121 - protein, 15 aa, Artificial Sequence
  LMP2a epitope
LTLESEERPPTPYRN

SEQ ID NO: 122 - protein, 15 aa, Artificial Sequence
  LMP2a epitope
LESEERPPTPYRNTV

SEQ ID NO: 123 - protein, 15 aa, Artificial Sequence
  EBNA-1 epitope
GGAGAGGGGRGRGGS

SEQ ID NO: 124 - protein, 15 aa, Artificial Sequence
  EBNA-1 epitope
AGGGGRGRGGSGGRG

SEQ ID NO: 125 - protein, 15 aa, Artificial Sequence
  EBNA-1 epitope
GRGRGGSGGRGRGGS

SEQ ID NO: 126 - protein, 15 aa, Artificial Sequence
  EBNA-1 epitope
GGSGGRGRGGSGGRG

SEQ ID NO: 127 - protein, 15 aa, Artificial Sequence
  EBNA-1 epitope
GRGRGGSGGRGRGGS

SEQ ID NO: 128 - protein, 15 aa, Artificial Sequence
  EBNA-1 epitope
GGSGGRGRGGSGGRR

SEQ ID NO: 129 - protein, 15 aa, Artificial Sequence
  EBNA-1 epitope
GRGRGGSGGRRGRGR

SEQ ID NO: 130 - protein, 15 aa, Artificial Sequence
  EBNA-1 epitope
GGSGGRRGRGRERAR

SEQ ID NO: 131 - protein, 15 aa, Artificial Sequence
  EBNA-1 epitope
GRRGRGRERARGGSR

SEQ ID NO: 132 - protein, 15 aa, Artificial Sequence
  EBNA-1 epitope
RGRERARGGSRERAR

SEQ ID NO: 133 - protein, 15 aa, Artificial Sequence
  EBNA-1 epitope
RARGGSRERARGRGR

SEQ ID NO: 134 - protein, 15 aa, Artificial Sequence
  EBNA-1 epitope
GSRERARGRGRGRGE

SEQ ID NO: 135 - protein, 15 aa, Artificial Sequence
  EBNA-1 epitope
RARGRGRGRGEKRPR

SEQ ID NO: 136 - protein, 15 aa, Artificial Sequence
  EBNA-1 epitope
RGRGRGEKRPRSPSS

SEQ ID NO: 137 - protein, 15 aa, Artificial Sequence
  EBNA-1 epitope
RGEKRPRSPSSQSSS

SEQ ID NO: 138 - protein, 15 aa, Artificial Sequence
  EBNA-1 epitope
RPRSPSSQSSSSGSP

SEQ ID NO: 139 - protein, 15 aa, Artificial Sequence
  EBNA-1 epitope
PSSQSSSSGSPPRRP

SEQ ID NO: 140 - protein, 15 aa, Artificial Sequence
  EBNA-1 epitope
SSSSGSPPRRPPPGR

SEQ ID NO: 141 - protein, 15 aa, Artificial Sequence
  EBNA-1 epitope
GSPPRRPPPGRRPFF

SEQ ID NO: 142 - protein, 15 aa, Artificial Sequence
  EBNA-1 epitope
RRPPPGRRPFFHPVG

SEQ ID NO: 143 - protein, 15 aa, Artificial Sequence
  EBNA-1 epitope
PGRRPFFHPVGDADY

SEQ ID NO: 144 - protein, 15 aa, Artificial Sequence
  EBNA-1 epitope
PFFHPVGDADYFEYL

SEQ ID NO: 145 - protein, 15 aa, Artificial Sequence
  EBNA-1 epitope
PVGDADYFEYLQEGG

SEQ ID NO: 146 - protein, 15 aa, Artificial Sequence
  EBNA-1 epitope
ADYFEYLQEGGPDGE

SEQ ID NO: 147 - protein, 15 aa, Artificial Sequence
  EBNA-1 epitope
EYLQEGGPDGEPDVP

SEQ ID NO: 148 - protein, 15 aa, Artificial Sequence
  EBNA-1 epitope
EGGPDGEPDVPPGAI

SEQ ID NO: 149 - protein, 15 aa, Artificial Sequence
  EBNA-1 epitope
DGEPDVPPGAIEQGP

SEQ ID NO: 150 - protein, 15 aa, Artificial Sequence
  EBNA-1 epitope
DVPPGAIEQGPTDDP

SEQ ID NO: 151 - protein, 15 aa, Artificial Sequence
  EBNA-1 epitope
GAIEQGPTDDPGEGP

SEQ ID NO: 152 - protein, 15 aa, Artificial Sequence
  EBNA-1 epitope
QGPTDDPGEGPSTGP

SEQ ID NO: 153 - protein, 15 aa, Artificial Sequence
  EBNA-1 epitope
DDPGEGPSTGPRGQG

SEQ ID NO: 154 - protein, 15 aa, Artificial Sequence
  EBNA-1 epitope
EGPSTGPRGQGDGGR

SEQ ID NO: 155 - protein, 15 aa, Artificial Sequence
  EBNA-1 epitope
TGPRGQGDGGRRKKG

SEQ ID NO: 156 - protein, 15 aa, Artificial Sequence
  EBNA-1 epitope
GQGDGGRRKKGGWFG

SEQ ID NO: 157 - protein, 15 aa, Artificial Sequence
  EBNA-1 epitope
GGRRKKGGWFGKHRG

SEQ ID NO: 158 - protein, 15 aa, Artificial Sequence
  EBNA-1 epitope
KKGGWFGKHRGQGGS

SEQ ID NO: 159 - protein, 15 aa, Artificial Sequence
  EBNA-1 epitope
WFGKHRGQGGSNPKF

SEQ ID NO: 160 - protein, 15 aa, Artificial Sequence
  EBNA-1 epitope
HRGQGGSNPKFENIA

SEQ ID NO: 161 - protein, 15 aa, Artificial Sequence
  EBNA-1 epitope
GGSNPKFENIAEGLR

SEQ ID NO: 162 - protein, 15 aa, Artificial Sequence
  EBNA-1 epitope
PKFENIAEGLRVLLA

SEQ ID NO: 163 - protein, 15 aa, Artificial Sequence
  EBNA-1 epitope
NIAEGLRVLLARSHV

SEQ ID NO: 164 - protein, 15 aa, Artificial Sequence
  EBNA-1 epitope
GLRVLLARSHVERTT

SEQ ID NO: 165 - protein, 15 aa, Artificial Sequence
  EBNA-1 epitope
LLARSHVERTTEEGN

SEQ ID NO: 166 - protein, 15 aa, Artificial Sequence
  EBNA-1 epitope
SHVERTTEEGNWVAG

SEQ ID NO: 167 - protein, 15 aa, Artificial Sequence
  EBNA-1 epitope
RTTEEGNWVAGVFVY

SEQ ID NO: 168 - protein, 15 aa, Artificial Sequence
  EBNA-1 epitope
EGNWVAGVFVYGGSK

SEQ ID NO: 169 - protein, 15 aa, Artificial Sequence
  EBNA-1 epitope
VAGVFVYGGSKTSLY

SEQ ID NO: 170 - protein, 15 aa, Artificial Sequence
  EBNA-1 epitope
FVYGGSKTSLYNLRR

SEQ ID NO: 171 - protein, 15 aa, Artificial Sequence
  EBNA-1 epitope
GSKTSLYNLRRGIAL

SEQ ID NO: 172 - protein, 15 aa, Artificial Sequence
  EBNA-1 epitope
SLYNLRRGIALAVPQ

SEQ ID NO: 173 - protein, 15 aa, Artificial Sequence
  EBNA-1 epitope
LRRGIALAVPQCRIT

SEQ ID NO: 174 - protein, 15 aa, Artificial Sequence
  EBNA-1 epitope
IALAVPQCRITPLSR

SEQ ID NO: 175 - protein, 15 aa, Artificial Sequence
  EBNA-1 epitope
VPQCRITPLSRLPFG

SEQ ID NO: 176 - protein, 15 aa, Artificial Sequence
  EBNA-1 epitope
RITPLSRLPFGMAPG

SEQ ID NO: 177 - protein, 15 aa, Artificial Sequence
  EBNA-1 epitope
LSRLPFGMAPGPGPQ

SEQ ID NO: 178 - protein, 15 aa, Artificial Sequence
  EBNA-1 epitope
PFGMAPGPGPQPGPL

SEQ ID NO: 179 - protein, 15 aa, Artificial Sequence
  EBNA-1 epitope
APGPGPQPGPLRESI

SEQ ID NO: 180 - protein, 15 aa, Artificial Sequence
  EBNA-1 epitope
GPQPGPLRESIVCYF

SEQ ID NO: 181 - protein, 15 aa, Artificial Sequence
  EBNA-1 epitope
GPLRESIVCYFMVFL

SEQ ID NO: 182 - protein, 15 aa, Artificial Sequence
  EBNA-1 epitope
ESIVCYFMVFLQTHI

SEQ ID NO: 183 - protein, 15 aa, Artificial Sequence
  EBNA-1 epitope
CYFMVFLQTHIFAEV

SEQ ID NO: 184 - protein, 15 aa, Artificial Sequence
  EBNA-1 epitope
VFLQTHIFAEVLKDA

SEQ ID NO: 185 - protein, 15 aa, Artificial Sequence
  EBNA-1 epitope
THIFAEVLKDAIKDL

SEQ ID NO: 186 - protein, 15 aa, Artificial Sequence
  EBNA-1 epitope
AEVLKDAIKDLVMTK

SEQ ID NO: 187 - protein, 15 aa, Artificial Sequence
  EBNA-1 epitope
KDAIKDLVMTKPAPT

SEQ ID NO: 188 - protein, 15 aa, Artificial Sequence
  EBNA-1 epitope
KDLVMTKPAPTCNIK

SEQ ID NO: 189 - protein, 15 aa, Artificial Sequence
  EBNA-1 epitope
MTKPAPTCNIKVTVC

SEQ ID NO: 190 - protein, 15 aa, Artificial Sequence
  EBNA-1 epitope
APTCNIKVTVCSFDD

SEQ ID NO: 191 - protein, 15 aa, Artificial Sequence
  EBNA-1 epitope
NIKVTVCSFDDGVDL

SEQ ID NO: 192 - protein, 15 aa, Artificial Sequence
  EBNA-1 epitope
TVCSFDDGVDLPPWF

SEQ ID NO: 193 - protein, 15 aa, Artificial Sequence
  EBNA-1 epitope
FDDGVDLPPWFPPMV

SEQ ID NO: 194 - protein, 15 aa, Artificial Sequence
  EBNA-1 epitope
VDLPPWFPPMVEGAA

SEQ ID NO: 195 - protein, 15 aa, Artificial Sequence
  EBNA-1 epitope
PWFPPMVEGAAAEGD

SEQ ID NO: 196 - protein, 15 aa, Artificial Sequence
  EBNA-1 epitope
PMVEGAAAEGDDGDD

SEQ ID NO: 197 - protein, 15 aa, Artificial Sequence
  EBNA-1 epitope
GAAAEGDDGDDGDEG

SEQ ID NO: 198 - protein, 15 aa, Artificial Sequence
  EBNA-1 epitope
EGDDGDDGDEGGDGD

SEQ ID NO: 199 - protein, 15 aa, Artificial Sequence
  EBNA-1 epitope
GDDGDEGGDGDEGEE

SEQ ID NO: 200 - protein, 15 aa, Artificial Sequence
  EBNA-1 epitope
GDEGGDGDEGEEGQE

(19) United States
(12) Patent Application Publication
(10) Pub. No.: US 2023/0210896 A9
(43) Pub. Date: Jul. 6, 2023

(54) METHOD FOR T CELL ACTIVATION FOR CANCER TREATMENT

(71) Applicant: GOOD T CELLS, INC., Seoul (KR)

(72) Inventors: Jung Ho Kim, Seoul (KR); Beom Seok Kim, Seoul (KR)

(73) Assignee: GOOD T CELLS, INC., Seoul (KR)

(21) Appl. No.: 17/429,235

(22) PCT Filed: Feb. 7, 2020
(86) PCT No.: PCT/KR2020/001734

§ 371 Date: Aug. 6, 2021

(30) Foreign Application Priority Data
Feb. 8, 2019 (KR) 10-2019-0015108

(51) Int. Cl. A61K 35/17; A61K 35/15; A61P 35/00; C12N 5/0781; C12N 5/0783; C12N 5/0784; C12N 5/0786; C07K 14/47

(52) U.S. Cl. CPC A61K 35/17; A61K 35/15; A61P 35/00; C12N 5/0635; C12N 5/0636; C12N 5/0639; C12N 5/0645; C07K 14/4748

(57) ABSTRACT

The antigen-presenting cell loaded with the cancer-specific tumor antigen epitope provided in the present invention, that is, a dendritic cell enables rapid and effective induction of differentiation and proliferation of cancer antigen-specific T cells, preferably memory T cells, and the memory T cells thus activated can treat a cancerous or neoplastic condition or prevent recurrence, progression, or metastasis of cancer while avoiding the defense mechanism of cancer cells.

### TECHNICAL FIELD

The present invention relates to a cancer-specific tumor antigen epitope, an antigen-presenting cell loaded with the epitope, and a method for activating T cells for cancer treatment using the antigen-presenting cell.

### BACKGROUND ART

Gastric cancer is known as a malignancy with a high incidence in the world, especially in Asia. There have been many known causes of development of gastric cancer; however, gastric cancer may be typically classified into EBV-associated gastric cancer, which is caused by infection with Epstein-Barr virus (EBV), and gastric cancer cell antigen-associated gastric cancer, which is caused by accumulation of genetic mutations in gastrointestinal cells. For current treatment for gastric cancer, excision of cancerous tissue has long been known to be the most effective, and chemotherapy and radiation therapy are also performed. However, it appears that gastric cancer is a hard-to-cure disease when not found early. In addition, although clinical trials have been conducted through several biological agents (antibodies, small molecules), therapeutic agents with good clinical effects have not yet been reported.

Recently, cancer cell-specific targeted therapy using patient-derived autologous T cells has been studied in several institutions, and clinical trials have been conducted for lymphoma using chimeric antigen receptor (CAR) T cells in several institutions. As a result, due to good clinical effects and low side effects, such therapy has attracted much attention as a new field of anticancer therapy.

Use of patient-derived T cells decreases induction of immune responses, which is the biggest side effect of cell therapeutic agents, and removes restrictions on the donor's HLA type. Thus, such T cells have been known as therapeutic agents which are effective and have no side effects. To date, CD8+ T cells, CD4+ T cells, NK cells, dendritic cells, and CAR T cells are known as types of cell therapeutic agents which are most widely used in the field of anticancer therapy. NK cells have cell-killing efficacy, and have several side effects due to not having antigen specificity. Dendritic cells are therapeutic agents belonging to the vaccine concept in that they have no function of directly killing cells, and are capable of delivering antigen specificity to T cells in the patient's body so that cancer cell specificity is imparted to T cells with high efficiency. In addition, CD4+ T cells play a role in helping other cells through antigen specificity, and CD8+ T cells are known to have the best antigen specificity and cell-killing effect.

However, most cell therapeutic agents, which have been used or developed to date, have limitations and thus have no clinical effect. Specifically, cancer cells, on their own, secrete substances that suppress immune responses in the human body, or do not present antigens necessary for production of antibodies against such cancer cells, thereby preventing an appropriate immune response from occurring.

Meanwhile, dendritic cells not only act as surveillants to detect antigens that come from the outside of the human body or are produced internally, but also quickly travel to the secondary lymphoid organs with such recognized and absorbed antigen, thereby acting as specialized antigen-presenting cells that present the antigens to immune cells, including T cells, which react with the antigens. Anti-cancer immunotherapeutic vaccines using dendritic cells have been developed through several methods, and may be largely divided into ex vivo generated dendritic cell vaccines and in vivo dendritic cell vaccines. The in vivo dendritic cell vaccine works in a manner of directly delivering an antigen to dendritic cells present in the body. In addition, a method using the ex vivo generated dendritic cell vaccine is in such a manner that dendritic cells are isolated from the patient's peripheral blood mononuclear cells (PBMCs) and an antigen to be presented is delivered to the isolated dendritic cells, through which the dendritic cells are activated and then injected back into the patient so that the antigen is delivered from the injected dendritic cells to T cells. In the latter, ex vivo dendritic cell culture method and antigen delivery method are important, and currently used antigen presentation methods include transfection of DNA of an antigen to be presented using virus or nucleofection, or antigen delivery targeting dendritic cells in which an antigen is bound to an antibody targeting the dendritic cells.

Currently, the biggest problems in dendritic cell vaccines are that severe chronic inflammatory phenomena in the body and the Warburg effect are exhibited, and it is considered very difficult to achieve effective activation of anticancer immune cells under the cancer microenvironment in which immunosuppressive cytokines, immunosuppressive T cells, dendritic cells, and the like are present.

### Technical Problem

An object of the present invention is to provide an Epstein-Barr virus (EBV)-positive cancer-specific tumor antigen epitope, and a composition for activating T cells which comprises the same.

Another object of the present invention is to provide an antigen-presenting cell loaded with the epitope of the present invention, the antigen-presenting cell being capable of activating T cells for cancer treatment.

Another object of the present invention is to provide a method for producing epitope-loaded antigen-presenting cells for cancer treatment.

Another object of the present invention is to provide a T cell, activated by the antigen-presenting cell loaded with the epitope of the present invention.

Still yet another object of the present invention is to provide a method for activating T cells for cancer treatment.

Still yet another object of the present invention is to provide a method for treating cancer using an epitope-loaded antigen-presenting cell.

Another object of the present invention is to provide a method for treating cancer using the T cells activated by the epitope-loaded antigen-presenting cells.

However, the technical problem to be solved by the present invention is not limited to the above-mentioned problems, and other problems which are not mentioned will be clearly understood by those skilled in the art from the following description.

### Solution to Problem

According to an embodiment of the present invention, there is provided a cancer-specific tumor antigen epitope.

In the present invention, the “cancer-specific tumor antigen epitope” is derived from a protein antigen which is present only in cancer cells and is not present in normal cells. In the present invention, the cancer-specific tumor antigen epitope includes at least one epitope recognized by T cell receptors; and such an epitope may preferably include epitopes present in Epstein-Barr virus (EBV)-positive cancer cells, including the EBV viral epitopes or cancer cell epitopes, and may more preferably be the Epstein-Barr virus (EBV)-positive cancer cell antigen which is Epstein-Barr virus latent membrane protein 2 (LMP2a), or Epstein-Barr nuclear antigen 1 (EBNA-1), or an epitope thereof.

In the present invention, the “Epstein-Barr virus latent membrane protein 2 (LMP2a)” is one of several EBV genes expressed in all of type II and III diseases/malignancies. LMP2a corresponds to a transmembrane protein that acts as a negative modulator of B cell-receptor signaling and promotes cell survival through sequestering of tyrosine kinases. HLA-A2-restricted peptides are epitope-specific cytotoxic T lymphocyte epitopes, which are detectable ex vivo in 60% to 75% of individuals, and are the most immunodominant LMP epitopes in latent diseases. The CLGGLLTMV peptide corresponds to a potential target for NPC and HL treatments, since the epitope is conserved in biopsies taken from NPC and HL patients and, along with other EBV latent epitopes, is immunologically weak.

In the present invention, the “Epstein-Barr nuclear antigen 1 (EBNA-1)” corresponds to a multifunctional, dimeric viral protein associated with Epstein-Barr virus. This corresponds to an EBV protein found only in all EBV-associated malignancies. This plays an important role in maintaining the altered state that cells take when the cells are infected with EBV. EBNA-1, on the other hand, corresponds to a glycine-alanine repeat sequence that separates a protein into amino- and carboxy-terminal domains. This sequence serves to stabilize a protein, prevent proteasomal breakdown, and impair antigen processing and MHC class I-restricted antigen presentation. Thus, the EBNA-1 inhibits CD8-restricted cytotoxic T cell responses against virus-infected cells. The EBNA-1 is expressed by Qp promoter in all latency programs and corresponds to the only viral protein expressed in latency program I.

In the present invention, the “epitope” refers to an epitope that is not present in a reference such as normal, non-cancerous cells or germline cells and is found in cancer cells. For example, the epitope may be, but is not limited to, a 10-mer to 20-mer, and preferably a 15-mer.

In the present invention, the epitope may exhibit binding affinity with at least one of HLA-A, HLA-B, HLA-C, HLA-E, HLA-F, HLA-G, β2-microglobulin, HLA-DPA1, HLA-DPB1, HLA-DQA1, HLA-DQB1, HLA-DRA1, HLA-DRB1, HLA-DRB3, HLA-DRB4, HLA-DRB5, HLA-DM, HLA-DOA, and HLA-DOB loci so that T cells, preferably memory T cells, extracted from human blood can have efficacy. Among these loci, the epitope may include those exhibiting high binding affinity with at least one of the HLA types that are most expressed by Koreans, for example, HLA-A*2402, HLA-A*A0201, HLA-A*3303, HLA-A*1101, HLA-A*0206, HLA-A*3101, HLA-B*5101, HLA-B*4403, HLA-B*5401, HLA-B*5801, and HLA-B*3501.

Preferably, in the present invention, the epitope has high binding affinity for HLA-A*2402 and may include epitopes of LMP2a antigen, each being a peptide represented by any one of SEQ ID NOs: 1 to 3, SEQ ID NOs: 7 to 9, SEQ ID NOs: 11 to 15, SEQ ID NOs: 19 to 21, SEQ ID NO: 23 to 39, SEQ ID NO: 43 to 45, SEQ ID NO: 47 to 51, SEQ ID NO: 55 to 57, SEQ ID NO: 59 to 63, SEQ ID NO: 67 to 69, SEQ ID NO: 71 to 75, SEQ ID NO: 79 to 81, SEQ ID NO: 83 to 111, SEQ ID NOs: 115 to 117, SEQ ID NOs: 119 to 122; or the epitope has high binding affinity for HLA-A*3101 and may include epitopes of EBNA-1 antigen, each being a peptide represented by any one of SEQ ID NOs: 124 to 127, SEQ ID NOs: 129 to 149, SEQ ID NOs: 151 to 154, SEQ ID NOs: 156 to 158, SEQ ID NOs: 160 to 163, SEQ ID NOs: 165 to 167, SEQ ID NOs: 168 to 176, SEQ ID NOs: 178 to 181, SEQ ID NOs: 183 to 194, and SEQ ID NOs: 196 to 199.

Here, in the present invention, for a method of measuring the epitope-HLA affinity, NetMHC 3.4 (URL: www.cbs.dtu.dk/services/NetMHC-3.4/) may be used to predict whether an epitope binds to a specific HLA allele. However, the present invention is not limited thereto.

In the present invention, the “HLA” or “human leukocyte antigen” refers to human gene that encodes a major histocompatibility complex (MHC) protein on the surface of cells that are responsible for regulation of the immune system. “HLA-I” or “HLA class I” refers to human MHC class I gene including HLA-A, HLA-B, HLA-C, HLA-E, HLA-F, HLA-G, and β2-microglobulin loci. “HLA-II” or “HLA class II” refers to human MHC class II gene including HLA-DPA1, HLA-DPB1, HLA-DQA1, HLA-DQB1, HLA-DRA1, HLA-DRB1, HLA-DRB3, HLA-DRB4, HLA-DRB5, HLA-DM, HLA-DOA, and HLA-DOB loci.

In the present invention, the cancer may be Epstein-Barr virus (EBV)-positive cancer, including EBV-positive gastric cancer, EBV-positive cervical cancer, EBV-positive Burkitt's lymphoma, EBV-positive T cell lymphoma, EBV-positive breast cancer, EBV-positive leiomyosarcoma, EBV-positive smooth muscle tumor, EBV-positive Hodgkin lymphoma, EBV-positive nasopharyngeal cancer, or EBV-positive post-transplant lymphoproliferative disorder (PTLD), with EBV-positive gastric cancer being preferred.

According to another embodiment of the present invention, there is provided a nucleic acid molecule, encoding a cancer-specific tumor antigen epitope provided in the present invention, preferably an epitope of LMP2a or EBNA-1 which is an antigen of Epstein-Barr virus (EBV)-positive cancer cells.

The nucleic acid molecule of the present invention includes any nucleic acid molecule obtained by converting an amino acid sequence of a polypeptide provided in the present invention into a polynucleotide sequence as known to those skilled in the art. Thus, various polynucleotide sequences may be prepared due to open reading frame (ORF), all of which are also included in the nucleic acid molecule of the present invention.

According to yet another embodiment of the present invention, there is provided an expression vector, into which the isolated nucleic acid molecule provided in the present invention is inserted.

In the present invention, the “vector” is a nucleic acid molecule which is capable of transporting another nucleic acid linked thereto. One type of vector is a “plasmid,” which refers to circular double-stranded DNA into which an additional DNA segment can be ligated. Another type of vector is a phage vector. Yet another type of vector is a viral vector, where an additional DNA segment can be ligated into the viral genome. Certain vectors are capable of autonomous replication in a host cell into which they are introduced (for example, bacterial vectors having a bacterial origin of replication and episomal mammalian vectors). Other vectors (for example, non-episomal mammalian vectors) can be integrated into the genome of a host cell upon introduction into the host cell, and thus are replicated along with the host genome. In addition, certain vectors are capable of directing expression of genes to which they are operatively linked. Such vectors are referred to herein as “recombinant expression vectors” or simply “expression vectors.” In general, expression vectors useful in recombinant DNA techniques are often in the form of plasmids. In the present specification, “plasmid” and “vector” may be used interchangeably as the plasmid is the most commonly used form among vectors.

Specific examples of the expression vector in the present invention may be selected from, but are not limited to, the group consisting of commercially widely used pCDNA vectors, F, R1, RP1, Col, pBR322, ToL, Ti vectors; cosmids; phages such as lambda, lambdoid, M13, Mu, p1 P22, Qμμ, T-even, T2, T3, T7; plant viruses. Any expression vector known, to those skilled in the art, as expression vectors can be used in the present invention, and the expression vector is selected depending on the nature of the target host cell. Introduction of a vector into a host cell may be performed by calcium phosphate transfection, viral infection, DEAE-dextran-mediated transfection, lipofectamine transfection, or electroporation. However, the present invention is not limited thereto, and those skilled in the art may adopt and use an introduction method appropriate for the expression vector and the host cell which are used. The vector may preferably contain at least one selection marker. However, the present invention is not limited thereto, and selection can be made using the vector that contains no selection marker, depending on whether or not a product is produced. The selection marker is selected depending on the target host cell, which is done using methods already known to those skilled in the art, and thus the present invention has no limitation thereon.

In order to facilitate purification of the nucleic acid molecule of the present invention, a tag sequence may be inserted into and fused to an expression vector. The tag includes, but is not limited to, hexa-histidine tag, hemagglutinin tag, myc tag, or flag tag, and any tag known to those skilled in the art which facilitates purification can be used in the present invention.

According to still yet another embodiment of the present invention, there is provided a host cell, transfected with the expression vector provided in the present invention.

In the present invention, the “host cell” includes individual cells or cell cultures which may be or have been recipients of the vector(s) for incorporation of a polypeptide insert. The host cell includes progeny of a single host cell, and the progeny may not necessarily be completely identical (in morphology or in genomic DNA complement) to the original parent cell due to natural, accidental, or intentional mutation. The host cell includes cells transfected in vivo with the polynucleotide(s) herein.

In the present invention, the host cell may include cells of mammalian, plant, insect, fungal, or cellular origin, and may be, for example, bacterial cells such as E. coli, Streptomyces, Salmonella typhimurium; fungal cells such as yeast cells and Pichia pastoris; insect cells such as Drosophila and Spodoptera Sf9 cells; animal cells such as Chinese hamster ovary (CHO) cells, SP2/0 (mouse myeloma), human lymphoblastoid, COS, NSO (mouse myeloma), 293T, Bowes melanoma cells, HT-1080, baby hamster kidney (BHK) cells, human embryonic kidney (HEK) cells, or PERC.6 (human retinal cells); or plant cells. However, the host cell is not limited thereto, and any cell known to those skilled in the art which can be used as a host cell is available.

According to still yet another embodiment of the present invention, there is provided a composition for activating T cells, comprising a cancer-specific tumor antigen epitope provided in the present invention, a nucleic acid molecule encoding the same, an expression vector into which the nucleic acid molecule is inserted, or a host cell transformed with the expression vector.

As used herein, “activation of T cells” refers to a population of monoclonal (for example, encoding the same TCR) or polyclonal (for example, having clones encoding different TCRs) T cells that have T cell receptors recognizing at least one tumor antigen peptide. Activated T cells may include one or more subtypes of T cells, including, but not limited to, one or more selected from the group consisting of cytotoxic T cells, helper T cells, natural killer T cells, γδ T cells, regulatory T cells, and memory T cells, with memory T cells being preferred.

In the present invention, the activated T cells can treat a cancerous or neoplastic condition or prevent recurrence, progression, or metastasis of cancer while avoiding the defense mechanism of cancer cells.

According to still yet another embodiment of the present invention, there is provided an antigen-presenting cell (APC) loaded with a cancer-specific tumor antigen epitope provided in the present invention.

In the present invention, the antigen-presenting cells may include at least one of dendritic cell (DC), B cell, and macrophage, with dendritic cell being preferred.

In the present invention, the antigen-presenting cells may be isolated from an individual.

In the present invention, the “dendritic cell” refers to any member of a diverse population of morphologically similar cell types found in lymphoid or non-lymphoid tissues. These cells are characterized by their distinctive morphology and high expression levels of surface class I and class II MHC molecules, which are proteins that present antigenic peptides to T cells. DCs, other APCs, and T cells may be isolated or derived (such as differentiated) from a number of tissue sources, and conveniently from peripheral blood, such as peripheral blood mononuclear cells (PBMCs) derived from peripheral blood.

In the present invention, the antigen-presenting cell can induce differentiation and proliferation of cancer antigen-specific T cells, preferably memory T cells, thereby treating a cancerous or neoplastic condition or preventing recurrence, progression, or metastasis of cancer while avoiding the defense mechanism of cancer cells.

According to still yet another embodiment of the present invention, there is provided a fusion protein, comprising: a cancer-specific tumor antigen epitope provided in the present invention; and a dendritic cell-specific antibody or a fragment thereof.

The fusion protein provided in the present invention enables the cancer-specific tumor antigen epitope provided in the present invention to be loaded on dendritic cells.

In the present invention, the dendritic cell-specific antibody may include, but is not limited to, antibodies specific for DCIR on dendritic cells, MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, Clec9A, 33D1, mannose receptor, Langerin, DECTIN-1, B7-1, B7-2, IFN-γ receptor, IL-2 receptor, ICAM-1, Fcγ receptor, LOX-1, or ASPGR.

The cancer-specific tumor antigen epitope in the fusion protein of the present invention may be conjugated to the dendritic cell-specific antibody or a fragment thereof. Here, the term “conjugate” refers to any material formed by joining two parts together. A representative conjugate according to the present invention includes those formed by joining an antigen together with an antibody and a TLR agonist. The term “conjugation” refers to a process of forming a conjugate and generally indicates physical coupling, for example, covalent bond, co-coordinate covalent bond, or second binding force, for example, Van der Waals binding force. The process of linking the antigen to the antibody may also be done via a non-covalent association such as a dockerin-cohesin association (as described in U.S. Patent Publication No. 20100135994, Banchereau et al. relevant portions incorporated herein by reference) or via a direct chemical linkage by forming a peptide or chemical bond.

According to another embodiment of the present invention, there is provided a method for producing an antigen-presenting cell, in which the antigen-presenting cell is loaded with a cancer-specific tumor antigen epitope provided in the present invention.

In the present invention, the antigen-presenting cell may include one or more of dendritic cell, B cell, and macrophage, with dendritic cell being preferred.

In the present invention, the dendritic cells (such as immature dendritic cells) may be obtained from a variety of sources including autologous sources, that is, derived from a target individual. The dendritic cells may preferably be obtained from peripheral blood mononuclear cells (PBMCs) derived from peripheral blood, and more preferably be obtained by isolating monocytes from individual-derived PBMCs and contacting the monocytes with a plurality of cytokines. Here, the type of cytokine that induces differentiation of the monocytes into dendritic cells is not particularly limited, and may include, for example, one or more of GM-CSF and IL-4.

In the present invention, the “target individual” means an individual who has or is at high risk of developing cancer.

In the present invention, once antigen-presenting cells are prepared as described above, the antigen-presenting cells may be loaded with a cancer-specific tumor antigen epitope of the present invention. In general, immature dendritic cells capture an antigen through phagocytosis or receptor-mediated endocytosis, process the antigen through a series of intracellular processes and then cause an antigenic peptide to be loaded on MHC and presented to T lymphocytes. With the process of processing an antigen, the dendritic cells become more mature, which makes them lose receptors used for phagocytosis and endocytosis, exhibit increased expression of MHC class I, II, costimulatory molecules, and adhesion molecules, and express new chemokine receptors. This allows the dendritic cells to migrate to T lymphocyte-rich areas of the surrounding lymph nodes, and to present the antigen to T lymphocytes, thereby causing a T lymphocyte immune response.

In an example of the present invention, in order for the cancer-specific tumor antigen epitope to be loaded on the antigen-presenting cell, the antigen-presenting cell may be contacted with the cancer-specific tumor antigen epitope of the present invention, and preferably, a step of pulsing, with the cancer-specific tumor antigen epitope of the present invention, the antigen-presenting cells, for example, immature dendritic cells, or antigen-presenting cells (such as dendritic cells) contained in or derived (for example, differentiated) from PBMCs may be performed. As known in the art, pulsing refers to a process of mixing cells, such as dendritic cells, with a solution containing a cancer-specific tumor antigen epitope peptide of the present invention, and then optionally removing the cancer-specific tumor antigen epitope peptide from the mixture. In the present invention, when the immature dendritic cells are contacted with the cancer-specific tumor antigen epitope, treatment with toll-like receptor agonists may be performed to further induce maturation of a population of immature dendritic cells. Here, exemplary TLR agonists include, but are not limited to, polylC, MALP, and R848.

In another example of the present invention, in order for the cancer-specific tumor antigen epitope to be loaded on the antigen-presenting cell, it is possible to perform nucleofection of the antigen-presenting cell with an expression vector, preferably a plasmid, into which a nucleic acid molecule encoding the cancer-specific tumor antigen epitope is inserted. Here, the nucleofection may be performed by any useful means in the art, including, for example, Amaxa® nucleofection system or InVitrogen® nucleofection system.

In yet another example of the present invention, in order for the cancer-specific tumor antigen epitope to be loaded on the antigen-presenting cell, such loading may be performed using a fusion protein that contains the cancer-specific tumor antigen epitope provided in the present invention; and a dendritic cell-specific antibody or a fragment thereof.

According to still yet another embodiment of the present invention, there is provided a T cell activated by an antigen-presenting cell provided in the present invention.

In the present invention, the T cells may be isolated from an individual.

In the present invention, the T cells refer to a population of monoclonal (for example, encoding the same TCR) or polyclonal (for example, having clones encoding different TCRs) T cells that have T cell receptors recognizing at least one tumor antigen peptide, and may include one or more subtypes of T cells, including, but not limited to, one or more selected from the group consisting of cytotoxic T cells, helper T cells, natural killer T cells, γδ T cells, regulatory T cells, and memory T cells, with memory T cells being preferred.

In the present invention, the “memory T cells” are T cells that have previously encountered and responded to their specific antigen, or T cells that have differentiated from activated T cells. Although tumor-specific memory T cells make up a small portion of the total T cell amount, they play an important function in surveillance of tumor cells during a person's entire lifespan. In a case where tumor-specific memory T cells encounter tumor cells that express their specific tumor antigen, the memory T cells are immediately activated and clonally expanded. The activated and expanded T cells differentiate into effector T cells to kill tumor cells with high efficiency. Memory T cells are important for establishing and maintaining long-term tumor antigen-specific responses of T cells. In the present invention, activated T cells, preferably activated memory T cells, specifically recognize antigens on cancer cells, so that such T cells can treat a cancerous or neoplastic condition or prevent recurrence, progression, or metastasis of cancer while avoiding the defense mechanism of cancer cells.

According to still yet another embodiment of the present invention, there is provided a method for activating T cells using an antigen-presenting cell (APC) provided in the present invention.

In the present invention, for activation of the T cells, the T cells may be co-cultured with antigen-presenting cells loaded with a cancer-specific tumor antigen epitope of the present invention.

In the present invention, the T cells may be obtained from various sources including autologous sources, that is, derived from a target individual, may preferably be obtained from peripheral blood mononuclear cells (PBMCs) derived from peripheral blood, and may more preferably be obtained from non-adherent portions of the peripheral blood mononuclear cells. In an example of the present invention, the non-adherent portions of the PBMCs may be obtained by density gradient centrifugation of a peripheral blood sample, or may be obtained by performing culture with at least one cytokine (such as IL-2) in the presence or absence of an anti-CD3 antibody (such as OKT3).

In the present invention, the T cells refer to a population of monoclonal (for example, encoding the same TCR) or polyclonal (for example, having clones encoding different TCRs) T cells that have T cell receptors recognizing a tumor antigen peptide, and may include one or more subtypes of T cells, including, but not limited to, one or more selected from the group consisting of cytotoxic T cells, helper T cells, natural killer T cells, γδ T cells, regulatory T cells, and memory T cells, with memory T cells being preferred.

In addition, in the present invention, the T cells and the antigen-presenting cells may be derived from the same individual, such as an individual suffering from cancer, preferably EBV-positive cancer (for example, low to medium grade cancer). However, the present invention is not limited thereto.

In the present invention, for activation of the T cells, the T cells may be co-cultured with antigen-presenting cells of the present invention for any one or more time periods of 1, 2, 3, 4, 6, 8, 10, 12, 14, 16, 18, 20, 22, 24, 26, 28, or 30 days, and preferably for 1 to 21 days, 1 to 14 days, 2 to 10 days, 2 to 5 days, 2 to 5 days, 3 days, 5 days, 7 days, 10 days, 14 days, 16 days, 18 days, or 21 days. However, the present invention is not limited thereto.

In the present invention, during the co-culture of the T cells with antigen-presenting cells of the present invention, one or more cytokines may be added to prime the T cells so that activation, maturation and/or proliferation of the T cells are promoted and the T cells subsequently differentiate into memory T cells. Exemplary cytokines that may be used at this stage include, but are not limited to, interleukin-2 (IL-2), interleukin-4 (IL-4), interleukin-7 (IL-7), interleukin-15 (IL-15), interleukin-21 (IL-21), or combinations thereof, and the like.

In addition, in the present invention, during the co-culture of the T cells with antigen-presenting cells of the present invention, a fusion protein comprising a cytokine and an immunoglobulin heavy chain constant region may be added to prime the T cells so that activation, maturation and/or proliferation of the T cells are promoted and the T cells subsequently differentiate into memory T cells. Here, the cytokine may include, but is not limited to, interferon-γ (IFN-γ), interleukin-2 (IL-2), interleukin-4 (IL-4), interleukin-12 (IL-12), IL-18, and tumor necrosis factor (TNF), or granulocyte macrophage colony stimulating factor (GMCSF). The immunoglobulin heavy chain constant region may also be, but is not limited to, an immunoglobulin hinge region, and an immunoglobulin heavy chain constant region optionally selected from the group consisting of CH2 domain, CH3 domain, and CH4 domain, or combinations thereof. In addition, the immunoglobulin heavy chain constant region may be derived from immunoglobulins belonging to any of five immunoglobulin classes called in the art as IgA (Igα), IgD (Igδ), IgE (Igε), IgG (Igγ), and IgM (Igμ), and may preferably be an immunoglobulin heavy chain constant region derived from the IgG class.

In addition, in the present invention, during the co-culture of the T cells with antigen-presenting cells of the present invention, a fusion protein that contains ligand binding to a cell surface protein which is highly expressed in memory T cells; and an immunoglobulin heavy chain constant region, may be added to prime the T cells so that activation, maturation and/or proliferation of the T cells are promoted and the T cells subsequently differentiate into memory T cells. Here, the cell surface protein which is highly expressed in memory T cells may be CD27, CXCR3, or CD62L. The ligand capable of binding to CD27 may be CD70; the ligand capable of binding to CXCR3 may be CXCR9 or CXCR10; and the ligand capable of binding to CD62L may be GlyCAM-1, CD34, MadCAM-1, or PSGL-1. However, the present invention is not limited thereto. In addition, the immunoglobulin heavy chain constant region may be derived from immunoglobulins belonging to any of five immunoglobulin classes called in the art as IgA (Igα), IgD (Igδ), IgE (Igε), IgG (Igγ), and IgM (Igμ), and may preferably be an immunoglobulin heavy chain constant region derived from the IgG class.

According to still yet another embodiment of the present invention, there is provided an immunotherapeutic agent, comprising, as an active ingredient, an antigen-presenting cell loaded with a cancer-specific tumor antigen epitope provided in the present invention. The immunotherapeutic agent according to the present invention can increase immune responses or may selectively increase some of immune responses desired for treatment or prevention of a certain disease, for example, cancer.

According to still yet another embodiment of the present invention, there is provided an anticancer vaccine or a pharmaceutical composition for preventing or treating cancer, comprising, as an active ingredient, an antigen-presenting cell loaded with a cancer-specific tumor antigen epitope provided in the present invention; and/or an activated T cell.

As used herein, the term “cancer” refers to or indicates a physiological condition characterized by cell growth in mammals which is not regulated in a typical manner. The cancer to be prevented, ameliorated, or treated in the present invention may be Epstein-Barr virus (EBV)-positive cancer, including, but not limited to, EBV-positive gastric cancer, EBV-positive cervical cancer, EBV-positive Burkitt's lymphoma, EBV-positive T cell lymphoma, EBV-positive breast cancer, EBV-positive leiomyosarcoma, EBV-positive smooth muscle tumor, EBV-positive Hodgkin lymphoma, EBV-positive nasopharyngeal cancer, or EBV-positive post-transplant lymphoproliferative disorder (PTLD), with EBV-positive gastric cancer being preferred.

The antigen-presenting cell provided in the present invention enables induction of differentiation and proliferation of EBV-positive cancer antigen-specific T cells, preferably memory T cells, and the memory T cells thus activated can treat a cancerous or neoplastic condition or prevent recurrence, progression, or metastasis of cancer while avoiding the defense mechanism of cancer cells.

The anticancer vaccine according to the present invention may involve both an immunization method performed by single administration and an immunization method performed by continuous administration.

In the present invention, the “prevention” may include, without limitation, any act of blocking symptoms of cancer, or suppressing or delaying the symptoms, using the pharmaceutical composition of the present invention.

In addition, in the present invention, the “treatment” may include, without limitation, any act of ameliorating or beneficially altering symptoms of cancer, using the pharmaceutical composition of the present invention.

In the present invention, the pharmaceutical composition may be characterized by being in the form of capsules, tablets, granules, injections, ointments, powders, or beverages, and the pharmaceutical composition may be characterized by being targeted to humans.

In the present invention, the pharmaceutical composition may be formulated in the form of oral preparations such as powders, granules, capsules, tablets, and aqueous suspensions, preparations for external use, suppositories, and sterile injectable solutions, respectively, according to conventional methods, and used. However, the pharmaceutical composition is not limited thereto. The pharmaceutical composition of the present invention may further comprise a pharmaceutically acceptable carrier. As the pharmaceutically acceptable carrier, a binder, a glidant, a disintegrant, an excipient, a solubilizer, a dispersant, a stabilizer, a suspending agent, a pigment, a flavor, and the like may be used for oral administration; a buffer, a preserving agent, a pain-relieving agent, a solubilizer, an isotonic agent, a stabilizer, and the like may be used in admixture for injections; and a base, an excipient, a lubricant, a preserving agent, and the like may be used for topical administration. The preparations of the pharmaceutical composition of the present invention may be prepared in various ways by being mixed with the pharmaceutically acceptable carrier as described above. For example, for oral administration, the pharmaceutical composition may be formulated in the form of tablets, troches, capsules, elixirs, suspensions, syrups, wafers, or the like. For injections, the pharmaceutical composition may be formulated in the form of unit dosage ampoules or multiple dosage forms. Alternatively, the pharmaceutical composition may be formulated into solutions, suspensions, tablets, capsules, sustained-release preparations, or the like.

Meanwhile, as examples of carriers, diluents, or excipients suitable for making preparations, lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, or the like may be used. In addition, a filler, an anti-coagulant, a lubricant, a wetting agent, a fragrance, an emulsifier, a preservative, and the like may further be included.

The route of administration of the pharmaceutical composition of the present invention includes, but is not limited to, oral, intravenous, intramuscular, intraarterial, intramedullary, intradural, intracardiac, transdermal, subcutaneous, intraperitoneal, intranasal, intestinal, topical, sublingual, or rectal route. Oral or parenteral administration is preferred.

As used herein, the term “parenteral” includes subcutaneous, intradermal, intravenous, intramuscular, intraarticular, intrabursal, intrasternal, intradural, intralesional, and intracranial injection or infusion techniques. The pharmaceutical composition of the present invention may also be administered in the form of suppositories for rectal administration.

The pharmaceutical composition of the present invention may vary depending on a variety of factors, including activity of a certain compound used, the patient's age, body weight, general health status, sex, diet, time of administration, route of administration, rate of excretion, drug combination, and severity of a certain disease to be prevented or treated. A dose of the pharmaceutical composition may vary depending on the patient's condition, body weight, severity of disease, drug form, route of administration, and duration, and may be appropriately selected by those skilled in the art. The pharmaceutical composition may be administered in an amount of 0.0001 to 50 mg/kg or 0.001 to 50 mg/kg, per day. Administration may be made once a day or several times a day. The dose is not intended to limit the scope of the present invention in any way. The pharmaceutical composition according to the present invention may be formulated in the form of pills, sugar-coated tablets, capsules, liquids, gels, syrups, slurries, or suspensions.

According to still yet another embodiment of the present invention, there is provided a method for preventing or treating cancer, comprising a step of administering, to a target individual, an antigen-presenting cell loaded with a cancer-specific tumor antigen epitope provided in the present invention; and/or an activated T cell.

In the present invention, the cancer may be Epstein-Barr virus (EBV)-positive cancer, including, but not limited to, EBV-positive gastric cancer, EBV-positive cervical cancer, EBV-positive Burkitt's lymphoma, EBV-positive T cell lymphoma, EBV-positive breast cancer, EBV-positive leiomyosarcoma, EBV-positive smooth muscle tumor, EBV-positive Hodgkin lymphoma, EBV-positive nasopharyngeal cancer, or EBV-positive post-transplant lymphoproliferative disorder (PTLD), with EBV-positive gastric cancer being preferred.

Dose, schedule, and route of administration of the antigen-presenting cell loaded with a cancer-specific tumor antigen epitope provided in the present invention or the activated T cell may be determined depending on the size and condition of an individual, and in accordance with standard pharmaceutical practice. Exemplary routes of administration include intravenous, intraarterial, intraperitoneal, intrapulmonary, intravascular, intramuscular, intratracheal, subcutaneous, intraocular, intrathecal, or transdermal route.

A dose of cells administered to an individual may vary depending, for example, on the particular type of cells being administered, the route of administration, and the particular type and stage of cancer being treated. The amount should be sufficient to produce a desirable response, such as a therapeutic response against cancer, but without severe toxicity or adverse events. In some embodiments, the amount of activated T cells or antigen-presenting cells (such as dendritic cells) to be administered is a therapeutically effective amount. In some embodiments, the amount of cells (such as dendritic cells loaded with a cancer-specific tumor antigen epitope or activated T cells) is an amount sufficient to decrease the size of a tumor, decrease the number of cancer cells, or decrease the growth rate of a tumor by any one of at least about 10%, 20%, 30%, 40%, 50%, 60%, 70%, 80%, 90%, 95%, or 100%, as compared with the corresponding tumor size, number of cancer cells, or tumor growth rate in the same individual prior to treatment or as compared with the corresponding activity in other individuals having not received the treatment. The magnitude of effects may be measured using standard methods, such as in vitro assays with purified enzymes, cell-based assays, animal models, or experiments using humans.

In an embodiment of the present invention, the antigen-presenting cells (such as dendritic cells) loaded with a cancer-specific tumor antigen epitope of the present invention may be administrated at a dose of any of 1×10⁵ to 5×10⁵, 5×10⁵ to 1×10⁶, 1×10⁶ to 2×10⁶, 2×10⁶ to 3×10⁶, 3×10⁶ to 4×10⁶, 4×10⁶ to 5×10⁶, 5×10⁶ to 6×10⁶, 6×10⁶ to 7×10⁶, 7×10⁶ to 8×10⁶, 8×10⁶ to 1×10⁸, 1×10⁶ to 3×10⁶, 3×10⁶ to 5×10⁶, 5×10⁶ to 7×10⁶, 2×10⁶ to 4×10⁶, 1×10⁶ to 5×10⁶, or 5×10⁶ to 1×10⁷ cells/individual. However, the present invention is not limited thereto.

In another embodiment of the present invention, the antigen-presenting cells (e.g., dendritic cells) loaded with a cancer-specific tumor antigen epitope of the present invention may be administrated at a dose of any of 1×10⁴ to 5×10⁴, 5×10⁴ to 1×10⁵, 1×10⁵ to 2×10⁵, 2×10⁵ to 4×10⁵, 4×10⁵ to 6×10⁵, 6×10⁵ to 8×10⁵, 8×10⁵ to 1×10⁶, 1×10⁶ to 2×10⁶, 2×10⁶ to 1×10⁷, 1×10⁴ to 1×10⁵, 1×10⁵ to 1×10⁶, 1×10⁶ to 1×10⁷, 1×10⁴ to 1×10⁶, or 1×10⁵ to 1×10⁷ cells/kg. However, the present invention is not limited thereto.

In addition, in an embodiment of the present invention, the activated T cells of the present invention may be administrated at a dose of any of 1×10⁸ to 5×10⁸, 5×10⁸ to 9×10⁸, 9×10⁸ to 1×10⁹, 1×10⁹ to 2×10⁹, 2×10⁹ to 3×10⁹, 3×10⁹ to 4×10⁹, 4×10⁹ to 5×10⁹, 5×10⁹ to 6×10⁹, 6×10⁹ to 1×10¹⁰, 1×10⁹ to 3×10⁹, 3×10⁹ to 5×10⁹, 5×10⁹ to 7×10⁹, 7×10⁹ to 1×10¹⁰, 1×10⁹ to 5×10⁹, 5×10⁹ to 1×10¹⁰, 3×10⁹ to 7×10⁹, 1×10¹⁰ to 1.5×10¹⁰, 1×10¹⁰ to 2×10¹⁰, or 1×10⁹ to 1×10¹⁰ cells/individual. However, the present invention is not limited thereto.

In another embodiment of the present invention, the activated T cells of the present invention may be administrated at a dose of any of 1×10⁷ to 1×10⁸, 1×10⁸ to 2×10⁸, 2×10⁸ to 4×10⁸, 4×10⁸ to 6×10⁸, 6×10⁸ to 8×10⁸, 8×10⁸ to 1×10⁹, 1×10⁹ to 2×10⁹, 2×10⁹ to 4×10⁹, 4×10⁹ to 1×10¹⁰, 2×10⁸ to 6×10⁸, 6×10⁸ to 1×10⁹, 1×10⁸ to 2×10⁸, 2×10⁸ to 2×10⁹, 1×10⁷ to 1×10⁸, 1×10⁸ to 1×10⁹, 1×10⁹ to 1×10¹⁰, or 1×10⁷ to 1×10⁹ cells/kg. However, the present invention is not limited thereto.

In the present invention, a stabilizer or excipient such as human albumin may be used together with administration of the antigen-presenting cells (such as dendritic cells) loaded with a cancer-specific tumor antigen epitope and/or the activated T cells.

In the present invention, dose and dosing schedule of the antigen-presenting cells (such as dendritic cells) loaded with a cancer-specific tumor antigen epitope and/or the activated T cells may be adjusted over the course of treatment based on the judgment of the administering physician. In some embodiments, the activated T cells may be administered at any time point of about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, or 1 month after the antigen-presenting cells loaded with a tumor antigen peptide are administered, or may be administered simultaneously with the antigen-presenting cells. However, the present invention is not limited thereto.

In the present invention, administration of the antigen-presenting cells (such as dendritic cells) loaded with a tumor specific epitope and/or the activated T cell may be done alone or in combination with other therapies, such as surgery, radiation therapy, gene therapy, immunotherapy, bone marrow transplantation, stem cell transplantation, hormone therapy, targeted therapy, cryotherapy, ultrasound therapy, photodynamic therapy, chemotherapy, or the like. Additionally, a person having a greater risk of developing a proliferative disease may receive treatments to inhibit and/or delay development of the disease.

### Advantageous Effects of Invention

The antigen-presenting cell, that is, dendritic cell, loaded with an Epstein-Barr virus (EBV)-positive cancer-specific tumor antigen epitope provided in the present invention enables rapid and effective induction of differentiation and proliferation of the cancer antigen-specific T cells, preferably memory T cells, and the memory T cells thus activated can treat an Epstein-Barr virus (EBV)-positive cancerous or neoplastic condition or prevent recurrence, progression, or metastasis of cancer while avoiding the defense mechanism of cancer cells.

In the conventional adoptive T cell therapies, it takes a long time of 3 to 6 months to produce a large number of T cells for treatment of cancer patients, which poses a big problem in the cell production process in immune cell therapy. However, according to the present invention, 10⁹ autologous memory T cells, which should be used for patient treatment, can be produced within three weeks, and cost reduction and minimized infection risk factors to external contaminants can be achieved. Accordingly, according to the present invention, there is provided a technique that can be applied to terminal cancer patients because such a technique makes rapid therapeutic approaches available for a larger number of solid cancer patients.

### BRIEF DESCRIPTION OF DRAWINGS

FIGS. 1 to 4 illustrate results obtained by performing ELISPOT on each of LMP2a-derived epitopes in order to check the degree of T cell activation caused by the epitope in normal human-derived PBMCs, according to an embodiment of the present invention.

FIGS. 5 to 8 illustrate results obtained by performing ELISPOT on each of EBNA-1-derived epitopes in order to check the degree of T cell activation caused by the epitope in normal human-derived PBMCs, according to an embodiment of the present invention.

### DETAILED DESCRIPTION OF INVENTION

According to an embodiment of the present invention, there is provided an Epstein-Barr virus (EBV)-positive cancer-specific tumor antigen epitope, represented by any one of SEQ ID NOs: 1 to 3, SEQ ID NOs: 7 to 9, SEQ ID NOs: 11 to 15, SEQ ID NOs: 19 to 21, SEQ ID NOs: 23 to 39, SEQ ID NOs: 43 to 45, SEQ ID NOs: 47 to 51, SEQ ID NOs: 55 to 57, SEQ ID NOs: 59 to 63, SEQ ID NOs: 67 to 69, SEQ ID NOs: 71 to 75, SEQ ID NOs: 79 to 81, SEQ ID NOs: 83 to 111, SEQ ID NOs: 115 to 117, SEQ ID NOs: 119 to 122, SEQ ID NOs: 124 to 127, SEQ ID NOs: 129 to 149, SEQ ID NOs: 151 to 154, SEQ ID NOs: 156 to 158, SEQ ID NOs: 160 to 163, SEQ ID NOs: 165 to 167, SEQ ID NOs: 168 to 176, SEQ ID NOs: 178 to 181, SEQ ID Nos: 183 to 194, and SEQ ID NOs: 196 to 199.

According to another embodiment of the present invention, there is provided an antigen-presenting cell (APC) loaded with a cancer-specific tumor antigen epitope provided in the present invention.

According to yet another embodiment of the present invention, there is provided a T cell activated by an antigen-presenting cell provided in the present invention.

According to still yet another embodiment of the present invention, there is provided an anticancer vaccine or a pharmaceutical composition for preventing or treating cancer, comprising, as an active ingredient, an antigen-presenting cell loaded with a cancer-specific tumor antigen epitope provided in the present invention; and/or an activated T cell.

According to still yet another embodiment of the present invention, there is provided a method for preventing or treating cancer, comprising administering, to an individual, an effective amount of an antigen-presenting cell loaded with a cancer-specific tumor antigen epitope provided in the present invention; and/or an activated T cell.

Hereinafter, the present invention will be described in more detail by way of examples. These examples are only for describing the present invention in more detail, and it will be apparent to those skilled in the art that according to the gist of the present invention, the scope of the present invention is not limited by these examples.

### EXAMPLES

[Example 1] Method for Producing EBV-Positive Gastric Cancer Cell Antigen Epitopes

Prediction of epitopes was made such that the epitope has 15-mer amino acids in the entire sequence of LMP2a or EBNA-1 as shown in Tables 1 and 2; and the epitopes in the sequence listing, which are set forth in Tables 1 (LMP2a) and 2 (EBNA-1), were produced according to a conventional method related to peptide production processes.

TABLE 1

| SEQ ID NO | Amino acid sequence |
|---|---|
| SEQ ID NO: 1 | MGSLEMVPMGAGPPS |
| SEQ ID NO: 2 | EMVPMGAGPPSPGGD |
| SEQ ID NO: 3 | MGAGPPSPGGDPDGD |
| SEQ ID NO: 4 | PPSPGGDPDGDDGGN |
| SEQ ID NO: 5 | GGDPDGDDGGNNSQY |
| SEQ ID NO: 6 | DGDDGGNNSQYPSAS |
| SEQ ID NO: 7 | GGNNSQYPSASGSYG |
| SEQ ID NO: 8 | SQYPSASGSYGNTPT |
| SEQ ID NO: 9 | SASGSYGNTPTPPND |
| SEQ ID NO: 10 | SYGNTPTPPNDEERE |
| SEQ ID NO: 11 | TPTPPNDEERESNEE |
| SEQ ID NO: 12 | PNDEERESNEEPPPP |
| SEQ ID NO: 13 | ERESNEEPPPPYEDL |
| SEQ ID NO: 14 | NEEPPPPYEDLDWGN |
| SEQ ID NO: 15 | PPPYEDLDWGNGDRH |
| SEQ ID NO: 16 | EDLDWGNGDRHSDYQ |
| SEQ ID NO: 17 | WGNGDRHSDYQPLGN |
| SEQ ID NO: 18 | DRHSDYQPLGNQDPS |
| SEQ ID NO: 19 | DYQPLGNQDPSLYLG |
| SEQ ID NO: 20 | LGNQDPSLYLGLQHD |
| SEQ ID NO: 21 | DPSLYLGLQHDGNDG |
| SEQ ID NO: 22 | YLGLQHDGNDGLPPP |
| SEQ ID NO: 23 | QHDGNDGLPPPPYSP |
| SEQ ID NO: 24 | NDGLPPPPYSPRDDS |
| SEQ ID NO: 25 | PPPPYSPRDDSSQHI |
| SEQ ID NO: 26 | YSPRDDSSQHIYEEA |
| SEQ ID NO: 27 | DDSSQHIYEEAGRGS |
| SEQ ID NO: 28 | QHIYEEAGRGSMNPV |
| SEQ ID NO: 29 | EEAGRGSMNPVCLPV |
| SEQ ID NO: 30 | RGSMNPVCLPVIVAP |
| SEQ ID NO: 31 | NPVCLPVIVAPYLFW |
| SEQ ID NO: 32 | LPVIVAPYLFWLAAI |
| SEQ ID NO: 33 | VAPYLFWLAAIAASC |
| SEQ ID NO: 34 | LFWLAAIAASCFTAS |
| SEQ ID NO: 35 | AAIAASCFTASVSTV |
| SEQ ID NO: 36 | ASCFTASVSTVVTAT |
| SEQ ID NO: 37 | TASVSTVVTATGLAL |
| SEQ ID NO: 38 | STVVTATGLALSLLL |
| SEQ ID NO: 39 | TATGLALSLLLLAAV |
| SEQ ID NO: 40 | LALSLLLLAAVASSY |
| SEQ ID NO: 41 | LLLLAAVASSYAAAQ |
| SEQ ID NO: 42 | AAVASSYAAAQRKLL |
| SEQ ID NO: 43 | SSYAAAQRKLLTPVT |
| SEQ ID NO: 44 | AAQRKLLTPVTVLTA |
| SEQ ID NO: 45 | KLLTPVTVLTAVVTF |
| SEQ ID NO: 46 | PVTVLTAVVTFFAIC |
| SEQ ID NO: 47 | LTAVVTFFAICLTWR |
| SEQ ID NO: 48 | VTFFAICLTWRIEDP |
| SEQ ID NO: 49 | AICLTWRIEDPPFNS |
| SEQ ID NO: 50 | TWRIEDPPFNSLLFA |
| SEQ ID NO: 51 | EDPPFNSLLFALLAA |
| SEQ ID NO: 52 | FNSLLFALLAAAGGL |
| SEQ ID NO: 53 | LFALLAAAGGLQGIY |
| SEQ ID NO: 54 | LAAAGGLQGIYVLVM |
| SEQ ID NO: 55 | GGLQGIYVLVMLVLL |
| SEQ ID NO: 56 | GIYVLVMLVLLILAY |
| SEQ ID NO: 57 | LVMLVLLILAYRRRW |
| SEQ ID NO: 58 | VLLILAYRRRWRRLT |
| SEQ ID NO: 59 | LAYRRRWRRLTVCGG |
| SEQ ID NO: 60 | RRWRRLTVCGGIMFL |
| SEQ ID NO: 61 | RLTVCGGIMFLACVL |
| SEQ ID NO: 62 | CGGIMFLACVLVLIV |
| SEQ ID NO: 63 | MFLACVLVLIVDAVL |
| SEQ ID NO: 64 | CVLVLIVDAVLQLSP |
| SEQ ID NO: 65 | LIVDAVLQLSPLLGA |
| SEQ ID NO: 66 | AVLQLSPLLGAVTVV |
| SEQ ID NO: 67 | LSPLLGAVTVVSMTL |
| SEQ ID NO: 68 | LGAVTVVSMTLLLLA |
| SEQ ID NO: 69 | TVVSMTLLLLAFVLW |
| SEQ ID NO: 70 | MTLLLLAFVLWLSSP |
| SEQ ID NO: 71 | LLAFVLWLSSPGGLG |
| SEQ ID NO: 72 | VLWLSSPGGLGTLGA |
| SEQ ID NO: 73 | SSPGGLGTLGAALLT |
| SEQ ID NO: 74 | GLGTLGAALLTLAAA |
| SEQ ID NO: 75 | LGAALLTLAAALALL |
| SEQ ID NO: 76 | LLTLAAALALLASLI |
| SEQ ID NO: 77 | AAALALLASLILGTL |
| SEQ ID NO: 78 | ALLASLILGTLNLTT |
| SEQ ID NO: 79 | SLILGTLNLTTMFLL |
| SEQ ID NO: 80 | GTLNLTTMFLLMLLW |
| SEQ ID NO: 81 | LTTMFLLMLLWTLVV |
| SEQ ID NO: 82 | FLLMLLWTLVVLLIC |
| SEQ ID NO: 83 | LLWTLVVLLICSSCS |
| SEQ ID NO: 84 | LVVLLICSSCSSCPL |
| SEQ ID NO: 85 | LICSSCSSCPLTKIL |
| SEQ ID NO: 86 | SCSSCPLTKILLARL |
| SEQ ID NO: 87 | CPLTKILLARLFLYA |
| SEQ ID NO: 88 | KILLARLFLYALALL |
| SEQ ID NO: 89 | ARLFLYALALLLLAS |
| SEQ ID NO: 90 | LYALALLLLASALIA |
| SEQ ID NO: 91 | ALLLLASALIAGGSI |
| SEQ ID NO: 92 | LASALIAGGSILQTN |
| SEQ ID NO: 93 | LIAGGSILQTNFKSL |
| SEQ ID NO: 94 | GSILQTNFKSLSSTE |
| SEQ ID NO: 95 | QTNFKSLSSTEFIPN |
| SEQ ID NO: 96 | KSLSSTEFIPNLFCM |
| SEQ ID NO: 97 | STEFIPNLFCMLLLI |
| SEQ ID NO: 98 | IPNLFCMLLLIVAGI |
| SEQ ID NO: 99 | FCMLLLIVAGILFIL |
| SEQ ID NO: 100 | LLIVAGILFILAILT |
| SEQ ID NO: 101 | AGILFILAILTEWGS |
| SEQ ID NO: 102 | FILAILTEWGSGNRT |
| SEQ ID NO: 103 | ILTEWGSGNRTYGPV |
| SEQ ID NO: 104 | WGSGNRTYGPVFMCL |
| SEQ ID NO: 105 | NRTYGPVFMCLGGLL |
| SEQ ID NO: 106 | GPVFMCLGGLLTMVA |
| SEQ ID NO: 107 | MCLGGLLTMVAGAVW |
| SEQ ID NO: 108 | GLLTMVAGAVWLTVM |
| SEQ ID NO: 109 | MVAGAVWLTVMTNTL |
| SEQ ID NO: 110 | AVWLTVMTNTLLSAW |
| SEQ ID NO: 111 | TVMTNTLLSAWILTA |
| SEQ ID NO: 112 | NTLLSAWILTAGFLI |
| SEQ ID NO: 113 | SAWILTAGFLIFLIG |
| SEQ ID NO: 114 | LTAGFLIFLIGFALF |
| SEQ ID NO: 115 | FLIFLIGFALFGVIR |
| SEQ ID NO: 116 | LIGFALFGVIRCCRY |
| SEQ ID NO: 117 | ALFGVIRCCRYCCYY |
| SEQ ID NO: 118 | VIRCCRYCCYYCLTL |
| SEQ ID NO: 119 | CRYCCYYCLTLESEE |
| SEQ ID NO: 120 | CYYCLTLESEERPPT |
| SEQ ID NO: 121 | LTLESEERPPTPYRN |
| SEQ ID NO: 122 | LESEERPPTPYRNTV |

TABLE 2

| SEQ ID NO | Amino acid sequence |
|---|---|
| SEQ ID NO: 123 | GGAGAGGGGRGRGGS |
| SEQ ID NO: 124 | AGGGGRGRGGSGGRG |
| SEQ ID NO: 125 | GRGRGGSGGRGRGGS |
| SEQ ID NO: 126 | GGSGGRGRGGSGGRG |
| SEQ ID NO: 127 | GRGRGGSGGRGRGGS |
| SEQ ID NO: 128 | GGSGGRGRGGSGGRR |
| SEQ ID NO: 129 | GRGRGGSGGRRGRGR |
| SEQ ID NO: 130 | GGSGGRRGRGRERAR |
| SEQ ID NO: 131 | GRRGRGRERARGGSR |
| SEQ ID NO: 132 | RGRERARGGSRERAR |
| SEQ ID NO: 133 | RARGGSRERARGRGR |
| SEQ ID NO: 134 | GSRERARGRGRGRGE |
| SEQ ID NO: 135 | RARGRGRGRGEKRPR |
| SEQ ID NO: 136 | RGRGRGEKRPRSPSS |
| SEQ ID NO: 137 | RGEKRPRSPSSQSSS |
| SEQ ID NO: 138 | RPRSPSSQSSSSGSP |
| SEQ ID NO: 139 | PSSQSSSSGSPPRRP |
| SEQ ID NO: 140 | SSSSGSPPRRPPPGR |
| SEQ ID NO: 141 | GSPPRRPPPGRRPFF |
| SEQ ID NO: 142 | RRPPPGRRPFFHPVG |
| SEQ ID NO: 143 | PGRRPFFHPVGDADY |
| SEQ ID NO: 144 | PFFHPVGDADYFEYL |
| SEQ ID NO: 145 | PVGDADYFEYLQEGG |
| SEQ ID NO: 146 | ADYFEYLQEGGPDGE |
| SEQ ID NO: 147 | EYLQEGGPDGEPDVP |
| SEQ ID NO: 148 | EGGPDGEPDVPPGAI |
| SEQ ID NO: 149 | DGEPDVPPGAIEQGP |
| SEQ ID NO: 150 | DVPPGAIEQGPTDDP |
| SEQ ID NO: 151 | GAIEQGPTDDPGEGP |
| SEQ ID NO: 152 | QGPTDDPGEGPSTGP |
| SEQ ID NO: 153 | DDPGEGPSTGPRGQG |
| SEQ ID NO: 154 | EGPSTGPRGQGDGGR |
| SEQ ID NO: 155 | TGPRGQGDGGRRKKG |
| SEQ ID NO: 156 | GQGDGGRRKKGGWFG |
| SEQ ID NO: 157 | GGRRKKGGWFGKHRG |
| SEQ ID NO: 158 | KKGGWFGKHRGQGGS |
| SEQ ID NO: 159 | WFGKHRGQGGSNPKF |
| SEQ ID NO: 160 | HRGQGGSNPKFENIA |
| SEQ ID NO: 161 | GGSNPKFENIAEGLR |
| SEQ ID NO: 162 | PKFENIAEGLRVLLA |
| SEQ ID NO: 163 | NIAEGLRVLLARSHV |
| SEQ ID NO: 164 | GLRVLLARSHVERTT |
| SEQ ID NO: 165 | LLARSHVERTTEEGN |
| SEQ ID NO: 166 | SHVERTTEEGNWVAG |
| SEQ ID NO: 167 | RTTEEGNWVAGVFVY |
| SEQ ID NO: 168 | EGNWVAGVFVYGGSK |
| SEQ ID NO: 169 | VAGVFVYGGSKTSLY |
| SEQ ID NO: 170 | FVYGGSKTSLYNLRR |
| SEQ ID NO: 171 | GSKTSLYNLRRGIAL |
| SEQ ID NO: 172 | SLYNLRRGIALAVPQ |
| SEQ ID NO: 173 | LRRGIALAVPQCRIT |
| SEQ ID NO: 174 | IALAVPQCRITPLSR |
| SEQ ID NO: 175 | VPQCRITPLSRLPFG |
| SEQ ID NO: 176 | RITPLSRLPFGMAPG |
| SEQ ID NO: 177 | LSRLPFGMAPGPGPQ |
| SEQ ID NO: 178 | PFGMAPGPGPQPGPL |
| SEQ ID NO: 179 | APGPGPQPGPLRESI |
| SEQ ID NO: 180 | GPQPGPLRESIVCYF |
| SEQ ID NO: 181 | GPLRESIVCYFMVFL |
| SEQ ID NO: 182 | ESIVCYFMVFLQTHI |
| SEQ ID NO: 183 | CYFMVFLQTHIFAEV |
| SEQ ID NO: 184 | VFLQTHIFAEVLKDA |
| SEQ ID NO: 185 | THIFAEVLKDAIKDL |
| SEQ ID NO: 186 | AEVLKDAIKDLVMTK |
| SEQ ID NO: 187 | KDAIKDLVMTKPAPT |
| SEQ ID NO: 188 | KDLVMTKPAPTCNIK |
| SEQ ID NO: 189 | MTKPAPTCNIKVTVC |
| SEQ ID NO: 190 | APTCNIKVTVCSFDD |
| SEQ ID NO: 191 | NIKVTVCSFDDGVDL |
| SEQ ID NO: 192 | TVCSFDDGVDLPPWF |
| SEQ ID NO: 193 | FDDGVDLPPWFPPMV |
| SEQ ID NO: 194 | VDLPPWFPPMVEGAA |
| SEQ ID NO: 195 | PWFPPMVEGAAAEGD |
| SEQ ID NO: 196 | PMVEGAAAEGDDGDD |
| SEQ ID NO: 197 | GAAAEGDDGDDGDEG |
| SEQ ID NO: 198 | EGDDGDDGDEGGDGD |
| SEQ ID NO: 199 | GDDGDEGGDGDEGEE |
| SEQ ID NO: 200 | GDEGGDGDEGEEGQE |

### [Example 2] ELISPOT Results for T Cells Activated by Dendritic Cells Loaded with Selected LMP2a Epitope

PBMCs extracted from the blood of three healthy humans (N5, N9, or N15) were separated into monocytes and leukocytes through magnetic-activated cell sorting, and the monocytes were cultured for 4 days in a culture supplemented with cytokines GM-CSF and IL-4 to differentiate into dendritic cells. To the culture, in which the monocytes had differentiated into dendritic cells, was added each group containing 10 or 11 epitope peptides as set forth in Table 3 together with poly(I:C) so that the respective peptides were transferred to the dendritic cells. The culture was matured for 1 day. In addition, the leukocytes were cultured for 3 days in a culture supplemented with anti-CD3/CD28 antibodies and cytokine IL-2.

TABLE 3

| Group | SEQ ID NO |
|---|---|
| 1 | SEQ ID NO: 1, SEQ ID NO: 13, SEQ ID NO: 25, SEQ ID NO: |
| | 37, SEQ ID NO: 49, SEQ ID NO: 61, SEQ ID NO: 73, SEQ ID |
| | NO: 85, SEQ ID NO: 97, SEQ ID NO: 109, SEQ ID NO: 121 |
| 2 | SEQ ID NO: 2, SEQ ID NO: 14, SEQ ID NO: 26, SEQ ID NO: |
| | 38, SEQ ID NO: 50, SEQ ID NO: 62, SEQ ID NO: 74, SEQ ID |
| | NO: 86, SEQ ID NO: 98, SEQ ID NO: 110, SEQ ID NO: 122 |
| 3 | SEQ ID NO: 3, SEQ ID NO: 15, SEQ ID NO: 27, SEQ ID NO: |
| | 39, SEQ ID NO: 51, SEQ ID NO: 63, SEQ ID NO: 75, SEQ ID |
| | NO: 87, SEQ ID NO: 99, SEQ ID NO: 111 |
| 4 | SEQ ID NO: 4, SEQ ID NO: 16, SEQ ID NO: 28, SEQ ID NO: |
| | 40, SEQ ID NO: 52, SEQ ID NO: 64, SEQ ID NO: 76, SEQ ID |
| | NO: 88, SEQ ID NO: 100, SEQ ID NO: 112 |
| 5 | SEQ ID NO: 5, SEQ ID NO: 17, SEQ ID NO: 29, SEQ ID NO: |
| | 41, SEQ ID NO: 53, SEQ ID NO: 65, SEQ ID NO: 77, SEQ ID |
| | NO: 89, SEQ ID NO: 101, SEQ ID NO: 113 |
| 6 | SEQ ID NO: 6, SEQ ID NO: 18, SEQ ID NO: 30, SEQ ID NO: |
| | 42, SEQ ID NO: 54, SEQ ID NO: 66, SEQ ID NO: 78, SEQ ID |
| | NO: 90, SEQ ID NO: 102, SEQ ID NO: 114 |
| 7 | SEQ ID NO: 7, SEQ ID NO: 19, SEQ ID NO: 31, SEQ ID NO: |
| | 43, SEQ ID NO: 55, SEQ ID NO: 67, SEQ ID NO: 79, SEQ ID |
| | NO: 91, SEQ ID NO: 103, SEQ ID NO: 115 |
| 8 | SEQ ID NO: 8, SEQ ID NO: 20, SEQ ID NO: 32, SEQ ID NO: |
| | 44, SEQ ID NO: 56, SEQ ID NO: 68, SEQ ID NO: 80, SEQ ID |
| | NO: 92, SEQ ID NO: 104, SEQ ID NO: 116 |
| 9 | SEQ ID NO: 9, SEQ ID NO: 21, SEQ ID NO: 33, SEQ ID NO: |
| | 45, SEQ ID NO: 57, SEQ ID NO: 69, SEQ ID NO: 81, SEQ ID |
| | NO: 93, SEQ ID NO: 105, SEQ ID NO: 117 |
| 10 | SEQ ID NO: 10, SEQ ID NO: 22, SEQ ID NO: 34, SEQ ID |
| | NO: 46, SEQ ID NO: 58, SEQ ID NO: 70, SEQ ID NO: 82, |
| | SEQ ID NO: 94, SEQ ID NO: 106, SEQ ID NO: 118 |
| 11 | SEQ ID NO: 11, SEQ ID NO: 23, SEQ ID NO: 35, SEQ ID |
| | NO: 47, SEQ ID NO: 59, SEQ ID NO: 71, SEQ ID NO: 83, |
| | SEQ ID NO: 95, SEQ ID NO: 107, SEQ ID NO: 119 |
| 12 | SEQ ID NO: 12, SEQ ID NO: 24, SEQ ID NO: 36, SEQ ID |
| | NO: 48, SEQ ID NO: 60, SEQ ID NO: 72, SEQ ID NO: 84, |
| | SEQ ID NO: 96, SEQ ID NO: 108, SEQ ID NO: 120 |
| 13 | SEQ ID NO: 1 to SEQ ID NO: 12 |
| 14 | SEQ ID NO: 13 to SEQ ID NO: 24 |
| 15 | SEQ ID NO: 25 to SEQ ID NO: 36 |
| 16 | SEQ ID NO: 37 to SEQ ID NO: 48 |
| 17 | SEQ ID NO: 49 to SEQ ID NO: 60 |
| 18 | SEQ ID NO: 61 to SEQ ID NO: 72 |
| 19 | SEQ ID NO: 73 to SEQ ID NO: 84 |
| 20 | SEQ ID NO: 85 to SEQ ID NO: 96 |
| 21 | SEQ ID NO: 97 to SEQ ID NO: 108 |
| 22 | SEQ ID NO: 109 to SEQ ID NO: 120 |
| 23 | SEQ ID NO: 121 and SEQ ID NO: 122 |

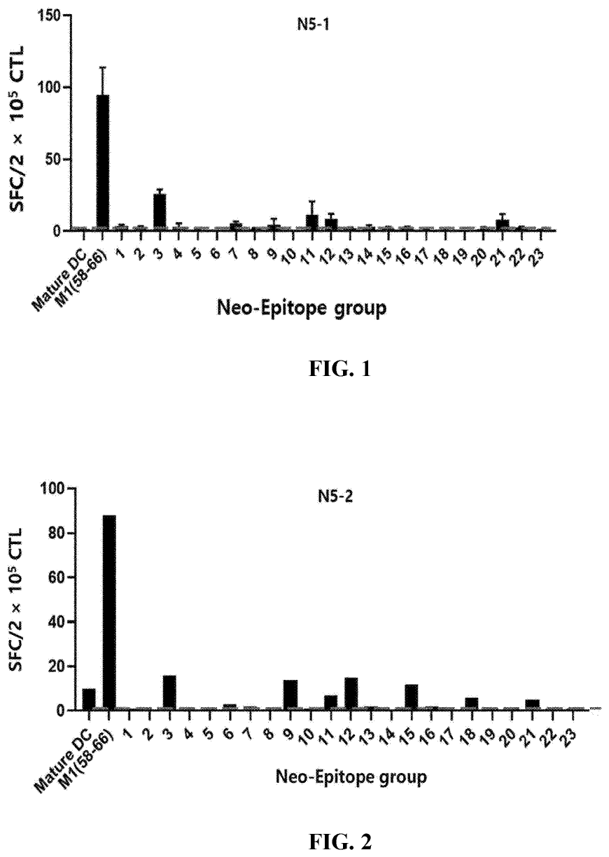

Then, CD8-expressing T cells were isolated from leukocytes, which were cultured in a culture supplemented with anti-CD3/CD28 antibody and cytokine IL-2, by magnetic-activated cell sorting, and co-cultured with mature dendritic cells at a ratio of 1:10 (dendritic cells:CD8-expressing T cells). In a case of the co-culture, the culture was mixed with a cytokine cocktail containing cytokines IL-4 and IL-7 that function to help survival and immune response of T cells, and the mixture was cultured. After 18 hours, secretion levels of IFN-γ in the T cells thus activated were measured with ELISPOT, and the results are illustrated in FIGS. 1 to 4. Here, as the positive control peptide, a peptide consisting of the sequence of 58^{th} to 66^{th} amino acids in the M1 protein, which is one of the proteins expressed by influenza virus, was employed.

As illustrated in FIGS. 1 and 2, for N5 (N5-1 and N5-2; the same experiment was repeated twice using the same person's blood), it was identified that the dendritic cells transformed with the epitope group 3, 7, 9, 11, 12, 15, 18, or 21 resulted in increased expression of IFN-γ in T cells.

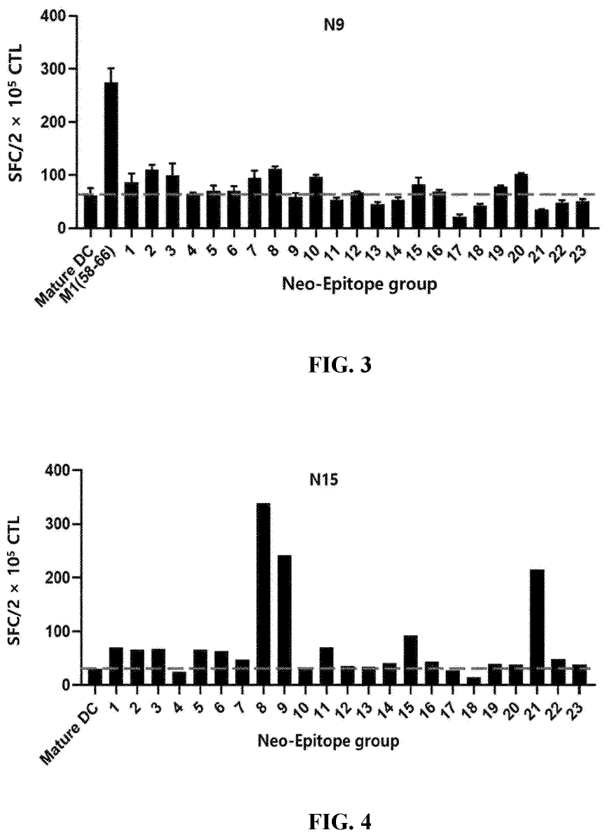

As illustrated in FIG. 3, for N9, it was identified that the dendritic cells transformed with the epitope group 1, 2, 3, 7, 8, 10, 15, 16, 19, or 20 resulted in increased expression of IFN-γ in T cells.

In addition, as illustrated in FIG. 4, for N15, it was identified that the dendritic cells transformed with the epitope group 1, 2, 3, 5, 6, 7, 8, 9, 11, 15, 21, or 22 resulted in increased expression of IFN-γ in T cells.

From these results, it was found that in the present invention, cytotoxic T lymphocytes (CTLs) can be activated by dendritic cells loaded with epitopes selected from the epitopes as set forth in Tables 1 and 2 above, and the thus activated T cells have antigen specificity which enables recognition of the epitope that is a novel antigen.

### [Example 3] ELISPOT Results for T Cells Activated with Dendritic Cells Loaded with Selected EBNA-1 Epitope

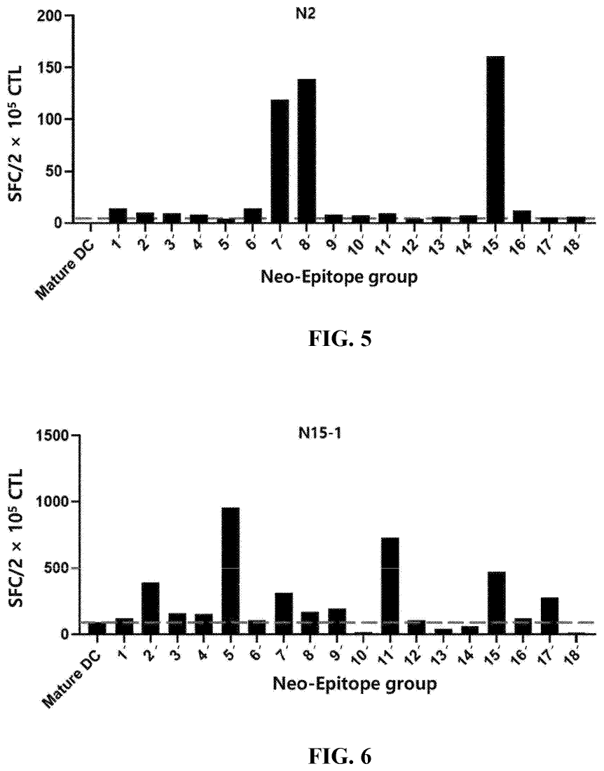

Using PBMCs extracted from the blood of three healthy humans (N2, N15, or N19), the degree of T cell activation was checked in the same manner as in Example 2, and the results are illustrated in FIGS. 5 to 8. Here, the monocytes having differentiated into dendritic cells were transformed with each group containing 10 or 11 epitope peptides as set forth in Table 4.

TABLE 4

| Group | SEQ ID NO |
|---|---|
| 1′ | SEQ ID NO: 123, SEQ ID NO: 132, SEQ ID NO: 141, SEQ |
| | ID NO: 150, SEQ ID NO: 159, SEQ ID NO: 168, SEQ ID NO: |
| | 177, SEQ ID NO: 186, SEQ ID NO: 195 |
| 2′ | SEQ ID NO: 124, SEQ ID NO: 133, SEQ ID NO: 142, SEQ |
| | ID NO: 151, SEQ ID NO: 160, SEQ ID NO: 169, SEQ ID NO: |
| | 178, SEQ ID NO: 187, SEQ ID NO: 196 |
| 3′ | SEQ ID NO: 125, SEQ ID NO: 134, SEQ ID NO: 143, SEQ |
| | ID NO: 152, SEQ ID NO: 161, SEQ ID NO: 170, SEQ ID NO: |
| | 179, SEQ ID NO: 188, SEQ ID NO: 197 |
| 4′ | SEQ ID NO: 126, SEQ ID NO: 135, SEQ ID NO: 144, SEQ |
| | ID NO: 153, SEQ ID NO: 162, SEQ ID NO: 171, SEQ ID NO: |
| | 180, SEQ ID NO: 189, SEQ ID NO: 198 |
| 5′ | SEQ ID NO: 127, SEQ ID NO: 136, SEQ ID NO: 145, SEQ |
| | ID NO: 154, SEQ ID NO: 163, SEQ ID NO: 172, SEQ ID NO: |
| | 181, SEQ ID NO: 190, SEQ ID NO: 199 |
| 6′ | SEQ ID NO: 128, SEQ ID NO: 137, SEQ ID NO: 146, SEQ |
| | ID NO: 155, SEQ ID NO: 164, SEQ ID NO: 173, SEQ ID NO: |
| | 182, SEQ ID NO: 191, SEQ ID NO: 200 |
| 7′ | SEQ ID NO: 129, SEQ ID NO: 138, SEQ ID NO: 147, SEQ |
| | ID NO: 156, SEQ ID NO: 165, SEQ ID NO: 174, SEQ ID NO: |
| | 183, SEQ ID NO: 192 |
| 8′ | SEQ ID NO: 130, SEQ ID NO: 139, SEQ ID NO: 148, SEQ |
| | ID NO: 157, SEQ ID NO: 166, SEQ ID NO: 175, SEQ ID NO: |
| | 184, SEQ ID NO: 193 |
| 9′ | SEQ ID NO: 131, SEQ ID NO: 140, SEQ ID NO: 149, SEQ |
| | ID NO: 158, SEQ ID NO: 167, SEQ ID NO: 176, SEQ ID NO: |
| | 185, SEQ ID NO: 194 |
| 10′ | SEQ ID NO: 123 to SEQ ID NO: 131 |
| 11′ | SEQ ID NO: 132 to SEQ ID NO: 140 |
| 12′ | SEQ ID NO: 141 to SEQ ID NO: 149 |
| 13′ | SEQ ID NO: 150 to SEQ ID NO: 158 |
| 14′ | SEQ ID NO: 159 to SEQ ID NO: 167 |
| 15′ | SEQ ID NO: 168 to SEQ ID NO: 176 |
| 16′ | SEQ ID NO: 177 to SEQ ID NO: 185 |
| 17′ | SEQ ID NO: 186 to SEQ ID NO: 194 |
| 18′ | SEQ ID NO: 195 to SEQ ID NO: 200 |

As illustrated in FIG. 5, for N2, it was identified that cytotoxic T lymphocytes were activated by the dendritic cells transformed with the epitope group 6′, 7′, 8′, or 15′.

In addition, as illustrated in FIGS. 6 and 7, for N15 (N15-1 and N15-2; the same experiment was repeated twice using the same person's blood), it was identified that cytotoxic T lymphocytes were activated by the dendritic cells transformed with each of all epitope groups except for some groups.

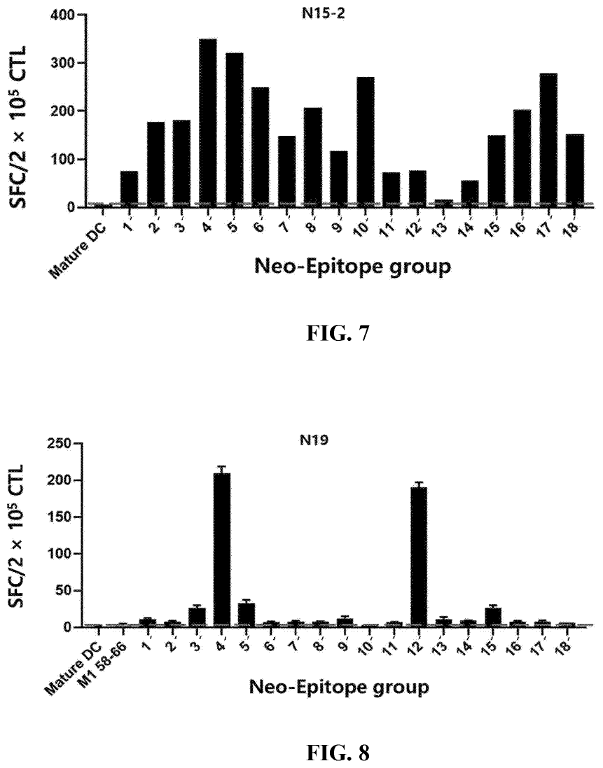

In addition, as illustrated in FIG. 8, for N19, it was identified that cytotoxic T lymphocytes were activated by the dendritic cells transformed with the epitope group 3′, 4′, 5′, 9′, 12′, or 15′.

From the results of Examples 2 and 3, it can be seen that the epitopes according to the present invention can very effectively activate cytotoxic T lymphocytes.

Although specific parts of the present invention have been described in detail, it is obvious to those skilled in the art that such a specific description is merely a preferred embodiment, and the scope of the present invention is not limited thereto. Therefore, the substantial scope of the present invention will be defined by the appended claims and equivalents thereof.

### INDUSTRIAL APPLICABILITY

The present invention relates to a cancer-specific tumor antigen epitope, an antigen-presenting cell loaded with the epitope, and a method for activating T cells for cancer treatment by the antigen-presenting cell.

1-36. (canceled)

37. An antigen-presenting cell loaded with an Epstein-Barr virus (EBV)-positive cancer-specific tumor antigen epitope, wherein the epitope is derived from Epstein-Barr virus latent membrane protein 2 (LMP2a) or Epstein-Barr nuclear antigen 1 (EBNA-1).

38. The antigen-presenting cell of claim 37, wherein the epitope is derived from LMP2a and comprises an amino acid sequence as set forth in any one of SEQ ID NOs: 1 to 3, SEQ ID NOs: 7 to 9, SEQ ID NOs: 11 to 15, SEQ ID NOs: 19 to 21, SEQ ID NO: 23 to 39, SEQ ID NO: 43 to 45, SEQ ID NO: 47 to 51, SEQ ID NO: 55 to 57, SEQ ID NO: 59 to 63, SEQ ID NO: 67 to 69, SEQ ID NO: 71 to 75, SEQ ID NO: 79 to 81, SEQ ID NO: 83 to 111, SEQ ID NOs: 115 to 117, and SEQ ID NOs: 119 to 122.

39. The antigen-presenting cell of claim 37, wherein the epitope is derived from EBNA-1 and comprises an amino acid sequence as set forth in any one of SEQ ID NOs: 124 to 127, SEQ ID NOs: 129 to 149, SEQ ID NOs: 151 to 154, SEQ ID NOs: 156 to 158, SEQ ID NOs: 160 to 163, SEQ ID NOs: 165 to 167, SEQ ID NOs: 168 to 176, SEQ ID NOs: 178 to 181, SEQ ID NOs: 183 to 194, and SEQ ID NOs: 196 to 199.

40. The antigen-presenting cell of claim 37, wherein the Epstein-Barr virus (EBV)-positive cancer is selected from the group consisting of EBV-positive gastric cancer, EBV-positive cervical cancer, EBV-positive Burkitt's lymphoma, EBV-positive T cell lymphoma, EBV-positive breast cancer, EBV-positive leiomyosarcoma, EBV-positive smooth muscle tumor, EBV-positive Hodgkin lymphoma, EBV-positive nasopharyngeal cancer, and EBV-positive post-transplant lymphoproliferative disorder (PTLD).

41. The antigen-presenting cell of claim 37, wherein the antigen-presenting cell promotes proliferation or differentiation of T cells and is selected from the group consisting of dendritic cell, B cell, and macrophage.

42. A method for producing the antigen-presenting cell of claim 37, comprising pulsing an antigen-presenting cell with the epitope.

43. The method of claim 42, wherein the antigen-presenting cell is mixed with a solution containing the epitope.

44. A method for producing the antigen-presenting cell of claim 37, comprising transfecting an antigen-presenting cell with an expression vector via nucleofection, wherein the expression vector comprises a nucleic acid sequence encoding the epitope.

45. A method for producing the antigen-presenting cell of claim 37, comprising loading an antigen-presenting cell with the epitope using a fusion protein that comprises the epitope.

46. The method of claim 45, wherein the fusion protein further comprises a dendritic cell-specific antibody or a fragment thereof.

47. The method of claim 46, wherein the epitope is conjugated to the dendritic cell-specific antibody or a fragment thereof.

48. The method of claim 46, wherein the dendritic cell-specific antibody is an antibody specific for DCIR, MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, Clec9A, 33D1, mannose receptor, Langerin, DECTIN-1, B7-1, B7-2, IFN-γ receptor, IL-2 receptor, ICAM-1, Fcγ receptor, LOX-1, or ASPGR.

49. A method for preventing or treating an Epstein-Barr virus (EBV)-positive cancer, the method comprising administering a therapeutically effective amount of the antigen-presenting cell of claim 37 to a target individual.

50. A method for activating T cells for cancer treatment or prevention, the method comprising co-culturing T cells with the antigen-presenting cell of claim 37.

51. The method of claim 50, wherein the T cells are obtained from peripheral blood mononuclear cells (PBMCs) of a target individual and include one or more selected from the group consisting of cytotoxic T cells, helper T cells, natural killer T cells, γδ T cells, regulatory T cells, and memory T cells.

52. The method of claim 50, wherein the co-culturing is performed with addition of interleukin-2 (IL-2), interleukin-4 (IL-4), interleukin-7 (IL-7), interleukin-15 (IL-15), interleukin-21 (IL-21), or a combination thereof.

53. The method of claim 50, wherein the co-culturing is performed with addition of a fusion protein that contains a cytokine and an immunoglobulin heavy chain constant region.

54. The method of claim 53, wherein the cytokine is interferon-γ (IFN-γ), interleukin-2 (IL-2), interleukin-4 (IL-4), interleukin-12 (IL-12), IL-18, tumor necrosis factor (TNF), or granulocyte macrophage colony stimulating factor (GMCSF).

55. The method of claim 50, wherein the co-culturing is performed with addition of a fusion protein that contains ligand of CD27, CXCR3, or CD62L, and an immunoglobulin heavy chain constant region.

56. A method for preventing or treating an Epstein-Barr virus (EBV)-positive cancer, the method comprising administering a therapeutically effective amount of T cells to a target individual, wherein the T cells are activated by the method of claim 50.
